SEQUENCE LISTING (45 sequences)

SEQ ID NO: 1 - protein, 17 aa, Homo sapiens
GIPFIETSTKTRQRVED

SEQ ID NO: 2 - protein, 17 aa, Homo sapiens
GIPFIETSTKTRQGVED

SEQ ID NO: 3 - protein, 17 aa, Homo sapiens
GIPFIETSVKTRQRVED

SEQ ID NO: 4 - protein, 17 aa, Homo sapiens
GIPFIETSVKTRQGVED

SEQ ID NO: 5 - protein, 17 aa, Homo sapiens
VPMVLVGNNCDLPSRTV

SEQ ID NO: 6 - protein, 17 aa, Homo sapiens
VPMVLVGNNCDLPTRTV

SEQ ID NO: 7 - protein, 30 aa, Homo sapiens
MTEYKLVVVGAGCVGKSALTIQLIQNHFVD

SEQ ID NO: 8 - protein, 30 aa, Homo sapiens
MTEYKLVVVGAGRVGKSALTIQLIQNHFVD

SEQ ID NO: 9 - protein, 30 aa, Homo sapiens
MTEYKLVVVGAGDVGKSALTIQLIQNHFVD

SEQ ID NO: 10 - protein, 30 aa, Homo sapiens
MTEYKLVVVGAGVVGKSALTIQLIQNHFVD

SEQ ID NO: 11 - protein, 30 aa, Homo sapiens
MTEYKLVVVGAGAVGKSALTIQLIQNHFVD

SEQ ID NO: 12 - protein, 30 aa, Homo sapiens
MTEYKLVVVGAGSVGKSALTIQLIQNHFVD

SEQ ID NO: 13 - protein, 30 aa, Homo sapiens
MTEYKLVVVGAAGVGKSALTIQLIQNHFVD

SEQ ID NO: 14 - protein, 30 aa, Homo sapiens
MTEYKLVVVGACGVGKSALTIQLIQNHFVD

SEQ ID NO: 15 - protein, 30 aa, Homo sapiens
MTEYKLVVVGADGVGKSALTIQLIQNHFVD

SEQ ID NO: 16 - protein, 30 aa, Homo sapiens
MTEYKLVVVGARGVGKSALTIQLIQNHFVD

SEQ ID NO: 17 - protein, 30 aa, Homo sapiens
MTEYKLVVVGASGVGKSALTIQLIQNHFVD

SEQ ID NO: 18 - protein, 30 aa, Homo sapiens
MTEYKLVVVGAVGVGKSALTIQLIQNHFVD

SEQ ID NO: 19 - protein, 30 aa, Homo sapiens
DGETCLLDILDTAGREEYSAMRDQYMRTGE

SEQ ID NO: 20 - protein, 30 aa, Homo sapiens
DGETCLLDILDTAGKEEYSAMRDQYMRTGE

SEQ ID NO: 21 - protein, 30 aa, Homo sapiens
DGETCLLDILDTAGHEEYSAMRDQYMRTGE

SEQ ID NO: 22 - protein, 30 aa, Homo sapiens
DGETCLLDILDTAGLEEYSAMRDQYMRTGE

SEQ ID NO: 23 - protein, 30 aa, Homo sapiens
DGETCLLDILDTAGEEEYSAMRDQYMRTGE

SEQ ID NO: 24 - protein, 30 aa, Homo sapiens
DGETCLLDILDTAGPEEYSAMRDQYMRTGE

SEQ ID NO: 25 - protein, 17 aa, Homo sapiens
KLVVVGAGCVGKSALTI

SEQ ID NO: 26 - protein, 17 aa, Homo sapiens
KLVVVGAGDVGKSALTI

SEQ ID NO: 27 - protein, 17 aa, Homo sapiens
KLVVVGAAGVGKSALTI

SEQ ID NO: 28 - protein, 17 aa, Homo sapiens
KLVVVGACGVGKSALTI

SEQ ID NO: 29 - protein, 17 aa, Homo sapiens
KLVVVGADGVGKSALTI

SEQ ID NO: 30 - protein, 17 aa, Homo sapiens
KLVVVGARGVGKSALTI

SEQ ID NO: 31 - protein, 17 aa, Homo sapiens
KLVVVGASGVGKSALTI

SEQ ID NO: 32 - protein, 17 aa, Homo sapiens
KLVVVGAVGVGKSALTI

SEQ ID NO: 33 - protein, 17 aa, Homo sapiens
KLVVVGAGRVGKSALTI

SEQ ID NO: 34 - protein, 17 aa, Homo sapiens
KLVVVGAGVVGKSALTI

SEQ ID NO: 35 - protein, 17 aa, Homo sapiens
LDILDTAGREEYSAMRD

SEQ ID NO: 36 - protein, 17 aa, Homo sapiens
LDILDTAGKEEYSAMRD

SEQ ID NO: 37 - protein, 17 aa, Homo sapiens
LDILDTAGHEEYSAMRD

SEQ ID NO: 38 - protein, 17 aa, Homo sapiens
LDILDTAGLEEYSAMRD

SEQ ID NO: 39 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 40 - protein, 21 aa, Homo sapiens
DDVPMVLVGNKCDLPSRTVDT

SEQ ID NO: 41 - protein, 21 aa, Homo sapiens
DDVPMVLVGNKCDLPTRTVDT

SEQ ID NO: 42 - protein, 21 aa, Homo sapiens
SYGIPFIETSAKTRQRVEDAF

SEQ ID NO: 43 - protein, 21 aa, Homo sapiens
SYGIPFIETSAKTRQGVEDAF

SEQ ID NO: 44 - protein, 189 aa, Homo sapiens [sequence omitted]

SEQ ID NO: 45 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

(19) United States
(12) Patent Application Publication
(10) Pub. No.: US 2019/0374628 A1
(43) Pub. Date: Dec. 12, 2019

(54) MUTATED FRAGMENTS OF THE RAS PROTEIN

(71) Applicant: Targovax ASA, Oslo (NO)

(72) Inventor: Jon Amund Eriksen, Porsgrunn (NO)

(21) Appl. No.: 15/736,472

(22) PCT Filed: Jun. 16, 2016
(86) PCT No.: PCT/EP2016/063920

§ 371 Date: Dec. 14, 2017

(30) Foreign Application Priority Data
Jun. 16, 2015 (EP) 15172418.4

(51) Int. Cl. A61K 39/00; A61P 35/00; A61P 37/00; A61K 35/17; C12N 9/14

(52) U.S. Cl. CPC A61K 39/001164; A61P 35/00; C12Y 306/05; A61K 35/17; C12N 9/14; A61P 37/00

(57) ABSTRACT

There is disclosed a peptide suitable for eliciting an immune response. The peptide corresponds to a fragment of the RAS protein, and comprises a region of 8 amino acids which includes a mutated position of the RAS protein. Said region has at least 6 amino acid residues, other than the mutated position, which are identical to the corresponding region of the RAS protein. The peptide has a point mutation at the amino acid corresponding to the mutated position, and the mutated position is position 146 or 117 of the RAS protein.

### FIELD OF THE INVENTION

The present invention provides peptides of the RAS protein for eliciting an immune response, peptide mixtures comprising peptides of the RAS protein for eliciting an immune response, T-cells specific for such peptides when presented on MHC molecules, and T-cell mixtures and T-cell preparations comprising T-cells specific for such peptides when presented on MHC molecules. The invention also relates to pharmaceutical formulations comprising such peptides, peptide mixtures, T-cells and T-cell mixtures and preparations, uses of such peptides, peptide mixtures, T-cells and T-cell mixtures and preparations for the prophylaxis and/or treatment of cancer, and methods of selecting peptides, peptide mixtures, T-cells, T-cell mixtures and T-cell preparations for the treatment of cancer

### BACKGROUND OF THE INVENTION

The genetic background for the onset of cancer is alterations in proto-oncogenes, oncogenes and tumour suppressor genes. Proto-oncogenes are normal genes of the cell which have the potential of becoming oncogenes. All oncogenes code for and function through a protein. In the majority of cases they have been shown to be components of signal transduction pathways. Oncogenes arise in nature from proto-oncogenes through point mutations or translocations, thereby resulting in a transformed state of the cell harbouring the mutation. Cancer develops through a multi-step process involving several mutational events in oncogenes and tumour suppressor cells.

In its simplest form, a single base substitution in a proto-oncogene may cause the encoded protein to differ in one amino acid.

In experimental models involving murine tumours, it has been shown that point mutations in intracellular “self”-proteins may give rise to tumour rejection antigens consisting of peptides differing in a single amino acid from the normal peptide. The T-cells recognizing these peptides in the context of major histocompatibility (MHC) molecules on the surface of the tumour cells are capable of killing the tumour cells and thus rejecting the tumour from the host. (Boon, T. et al, Cell 1989, Vol. 58, p. 293-303) In the last three decades, particular effort has been devoted to the analysis of antibodies to human tumour antigens. It has been suggested that such antibodies could be used both for diagnostic and therapeutic purposes, for instance in connection with an anti-cancer agent. One problem is that antibodies can only bind to tumour antigens that are exposed on the surface of tumour cells. For this reason the efforts to produce a cancer treatment based on the immune system of the body has been less successful than expected.

Antibodies typically recognise free antigens in native conformation and can potentially recognise almost any site exposed on the antigen surface. In contrast to the antibodies produced by the B cells, T-cells recognise antigens only in the context of MHC molecules, designated HLA (human leukocyte antigen) in humans, and only after appropriate antigen processing, usually consisting of proteolytic fragmentation of the protein, resulting in peptides that fit into the groove of the MHC molecules. This enables T-cells to recognise peptides derived from intracellular proteins. T-cells can thus recognise aberrant peptides derived from anywhere in the tumour cell, when displayed on the surface of the tumour cell by MHC molecules. The T-cell can subsequently be activated to eliminate the tumour cell harbouring the aberrant peptide.

T-cells may control the development and growth of cancer by a variety of mechanisms. Cytotoxic T-cells, both HLA class I restricted CD8+ and HLA Class II restricted CD4+, may directly kill tumour cells carrying the appropriate tumour antigens. CD4+ helper T-cells are needed for induction and maintenance of cytotoxic T-cell responses as well as for antibody responses, and for inducing macrophage and lymphokine-activated killer cell (LAK cell) killing.

Many oncogenes and their protein products have been identified. In addition, it has been shown that the T-cell repertoire of a healthy person includes T-cells with specificity against a synthetic peptide fragment derived from one p21 RAS oncogene product, when presented on an appropriate HLA molecule. Furthermore, it is anticipated that approximately 20% of all cancers are associated with a mutation in the RAS oncogene.

Epidermal growth factor receptor (EGFR) antibody therapy is a first-line regimen for the treatment of cancer. However, it has been found that, in colorectal cancer, point mutations in codons 12 and 13 of exon 2 of the KRAS gene are predictive of resistance to treatment with EGFR antibodies, such that treatment with EGFR antibodies is ineffective for patients having tumours expressing such mutations. It has also been found that more than half of patients who have tumours expressing wild-type KRAS codons 12 and/or 13 are also resistant to EGFR antibody therapy (Vaughn C P et al., Genes Chromosomes Cancer, 2011, 50, 307-12). Furthermore, it has been reported that patients having any RAS mutation may even be harmed by paitumumab-FOLFOX4 treatment (Douillard J Y et al., N Eng J Med 2013, 369, 1023-34). In particular, current clinical guidelines recommend that only patients with RAS wild-type tumours should be treated with cetuximab or panitumumab in combination with FOLFIRI or FOLFOX chemotherapy (Van Cutsem et al., JCO, 2015 Jan. 20; National Comprehensive Cancer Network: NCCN Guidelines Colon cancer, version 2.2015).

Van Cutsem et al. (JCO, published online ahead of print on 20 Jan. 2015) discloses the results of a study assessing the effect of FOLFIRI plus cetuximab compared with FOLFIRI alone in colorectal cancer patients carrying tumours having RAS mutations at locations other than in KRAS codon 12 and 13. It was found that the most common site of mutation (outside of KRAS codons 12 and 13) was within KRAS exon 4, and that the addition of cetuximab to FOLFIRI did not provide any improvement in efficacy of treatment in patients with tumour RAS mutations in loci other than codons 12 and 13. However, this document does not disclose or suggest any alternative treatments for patients having RAS mutations.

Sorich et al. (Annals of Oncology, 26: 13-21, 2015) reports a study to evaluate the efficacy of anti-EGFR mAbs therapy in metastatic colorectal cancer patients having tumours with NRAS or KRAS mutations outside of exon 2 (referred to as the “new” RAS mutations). The “new” mutations considered were KRAS mutations in exon 3 (codons 59 and 61) or exon 4 (codons 117 and 146), or NRAS mutations in exon 2, 3 or 4. It was found that there was no improvement in progression-free survival (PFS) or overall survival (OS) from anti-EGFR mAb therapy in patients having the “new” RAS mutations, and that patients having wild-type RAS had a significantly superior anti-EGFR mAb efficacy compared with patients having “new” RAS mutations. This document further discloses that about 11% of colorectal cancer patients have a “new” RAS mutation and, therefore, are likely to be resistant to EGFR antibody therapy. However, this document does not suggest any alternative treatments to EGFR antibody therapy.

Negru et al. (BMJ Open 2014, 4) discloses that anti-EGFR mAb therapy is ineffective in patients having mutations in the RAS gene, and reports a study to develop and validate a high-resolution melting (HRM) method for the detection of KRAS and NRAS mutations in colorectal cancer patients. This document discloses that 15.31% of colorectal cancer patients studied, having wild-type KRAS exon 2, had a mutation in KRAS exons 3 or 4, or NRAS exons 2, 3 or 4. In particular, exon 4 mutations accounted for 5.3% of RAS mutations outside of exon 2 in the patients studied, as compared to 1.91% for each of KRAS and NRAS exon 3. However, this document does not suggest alternative treatments to EGFR antibody therapy for patients having RAS mutations and, in particular, for patients having RAS mutations other than RAS exon 2 mutations.

Prior to the Van Cutsem et al., Sorrich et al. and Negru et al., it was not known that mutations in exon 4 of the RAS protein were associated with cancer. Thus, peptides corresponding to exon 4 of the RAS protein have not been proposed or tested for their usefulness in treating cancer.

WO 92/14756 discloses synthetic peptides and fragments of oncogene protein products which elicit T-cell immunity, for use in vaccines against cancers associated with RAS and compositions for the treatment of cancer. The peptides must correspond to an active fragment of the oncogene as presented by the cancer cell and include a mutation in one or more positions corresponding to the oncogene mutation. This document discloses mutations at positions 12, 13 and 61 of the RAS protein and specifically discloses only G12A, G12V, G12C, G12S, G12K, G12D, G12R, Q61R, Q61K, Q61L, Q61H, G13V and G13D mutations. However, this document does not disclose using any peptides having mutations in codons other than 12, 13 and 61 and, in particular, using peptides corresponding to RAS exon 4. In addition, while this document mentions that vaccines may comprise a selection of peptides having the most common mutations found in oncogene proteins, it does not suggest any specific combinations.

WO 00/66153 discusses synthetic peptide mixtures which elicit T-cell immunity for use in cancer vaccines. The peptide mixtures consist of RAS p21 mutant peptides and this document specifically discloses only G12A, G12C, G12D, G12R, G12S, G12V, Q61H, Q61K, Q61L, Q61R and G13D mutations. This document also discloses that the immune response elicited by a cocktail of peptides was significantly higher than that elicited by a single peptide; however, it does not suggest any RAS peptides having mutations other than at codons 12, 13 or 61 or, in particular, in exon 4. In addition, this document does not disclose that any other combinations of peptides other than those specifically disclosed therein may be useful.

GB 2328689 discloses that a peptide capable of inducing specific cytotoxic T-cell responses (CD 8+) comprises 8 to 10 amino acids of the p21 ras proto-oncogene protein including position 12 and/or 13, or position 61, of the p21 RAS proto-oncogene protein and have an amino acid substitution in position 12, 13 or 61. This document also discloses that the peptide may be used as a cancer vaccine and in compositions for anti-cancer treatment. However, peptides other than those having mutations at position 12, 13 or 61 are not disclosed, and no specific peptide mixtures are disclosed as being particularly useful.

Furthermore, none of these documents discusses how particular peptides are associated with particular types of cancer.

There are great concerns about using peptide mixtures for vaccination of patients due to the risk that some of the peptides in the mixture are immunodominant and thus suppress the HLA presentation of the other peptides (Pion S, et al., Blood, 1999 Feb. 1; 93(3): p 952-62). From experiments performed in vitro, it is known that various mutated RAS peptides may compete for binding to the HLA molecule responsible for presentation to the relevant T-cells and that peptides of the same length, but representing different mutations may inhibit the binding and recognition of a peptide representing another mutation with different degrees of efficacy (T. Gedde-Dahl III et al., Human Immunol. 1994, 33, p. 266-274, and B. H. Johanssen et al., Scand. J. Immunol., 1994, 33, p. 607-612). From these facts, the immunodominance issue has been regarded as a problem for mutated RAS peptide vaccines, but this issue is not discussed in any of the documents mentioned in the preceding paragraphs. Gjertsen et al. (Int. J. Cancer 2001, 92, p. 441-450) discloses a phase I/11 trial involving patients with adenocarcinoma of the pancreas vaccinated with synthetic mutant RAS peptides in combination with granulocyte-macrophage colony-stimulating factor. This trial used single peptide vaccines or a mixture of four mutant peptides. The combination vaccine consisted of the four most common KRAS mutations found in pancreatic adenocarcinoma, namely peptides having a G12V, a G12D, a G12C or a G12R mutation. However, this document does not disclose any other combinations of peptides that may be useful, does not disclose any other mutations of the RAS protein that are associated with cancer, does not discuss how particular peptides are associated with particular types of cancer and does not discuss the issue of immunodominance and redundancy within a vaccine.

Weden et al. (Int. J. Cancer 2010, 128(5), p. 1120-1128) reports the long-term follow-up of patients with pancreatic adenocarcinoma vaccinated with synthetic mutant RAS peptides. The vaccine consisted of either a single RAS peptide or a cocktail of seven RAS peptides. In particular, the seven RAS peptides used in this trial had a G12A, a G12C, a G12D, a G12R, a G12S, a G12V or a G13D mutation. However, this document does not discuss how particular peptides are associated with particular types of cancer, does not disclose any other combinations of peptides which may be useful, and does not discuss the issue of immunodominance and redundancy within a vaccine.

Hunger et al. (Exp. Dermatol. 2001, 10: 161-167) reports a clinical pilot study of the in vivo immunogenicity of RAS peptides with safety as the primary end point and immunogenicity of RAS peptides as a secondary end point. Melanoma patients were immunised intradermally with N-ras peptides (residue 49-73) with four codon 61 mutations. Eight of the patients showed positive DTH responses. However, this document does not discuss how particular peptides are associated with particular types of cancer, does not disclose any other combinations of peptides which may be useful, and does not discuss the issue of immunodominance and redundancy within a vaccine.

Prior et al. (Cancer Res. 2012, 72(10), p. 2457-2467) discloses that different types of cancer are coupled to mutation of a particular RAS isoform and that each isoform has a distinctive codon mutation signature. In addition, Prior et al. discloses that a total of 18 mutations occur in positions 12, 13 and 61 of the RAS protein, with six mutations occurring in each position. This review also discusses the effects of these mutations on RAS function and the potential mechanisms leading to differential patterns of RAS isoform mutations. However, this document does not address the treatment or prophylaxis of cancer, or the issue of immunodominance and redundancy within a vaccine. In addition, there is no disclosure of a vaccine or treatment against cancer, and this document does not disclose any combinations of peptides which may be useful.

Thus, there is a need to provide an alternative treatment to EGFR antibody therapy, which can be administered to cancer patients carrying tumours with RAS mutations. In particular, it is desirable to provide an alternative treatment to EGFR antibody therapy for patients carrying tumours having RAS exon 4 mutations, and especially for patients having colorectal cancer. In addition, there is a need to provide alternative treatments which are targeted to particular cancers, which overcome the issue of immunodominance and redundancy, and which are cost-effective. Furthermore, there is a need to provide vaccines and/or treatments for as many colorectal cancer patients as possible.

### SUMMARY OF THE INVENTION

The present invention provides solutions to the problems discussed above because it has now been found that peptides having a point mutation at an amino acid corresponding to a codon of exon 4 of the RAS gene, and peptide mixtures comprising at least one such peptide, can be used as a vaccine against and/or as a treatment for cancers associated with a RAS protein mutation. In particular, it has been found that at least some of the peptide mixtures of the present invention can be used as vaccines against and/or treatments for over 99% of cancers associated with mutations in RAS proteins, and as vaccines against and/or treatments for 10% of all colorectal cancers. In particular, when considered in combination with the peptide mixture known as TG02 (discussed further herein), the peptide and the peptide mixtures of the present invention can be used to treat at least 50% of all colorectal cancers. The peptides and peptide mixtures of the present invention provide an alternative treatment for patients resistant to EGFR antibody therapy. Further, the peptides and peptide mixtures of the present invention alleviate issues of immunodominance and reduce the redundancy of active ingredients within a pharmaceutical composition, thus making the peptides and peptide mixtures more cost-effective vaccines and/or treatments. In addition, the present invention allows for vaccination and/or treatment that is targeted to specific types of cancer and methods of selecting mixtures of peptides targeted to specific types of cancer.

Thus, in a first aspect of the invention, there is provided a peptide suitable for eliciting an immune response, wherein said peptide comprises a region which corresponds to a fragment of the RAS protein, wherein
said region comprises at least 8 amino acids which include a mutated position,
said region has at least 6 amino acid residues, other than at said mutated position, which are identical to the corresponding region of the RAS protein,
said region has a point mutation at said mutated position, and
said mutated position is position 117 or 146 of the RAS protein.

Advantageously, the point mutation is a K117N, a A146T or a A146V mutation.

Preferably, the peptide is for use as a vaccine or medicament.

In second aspect of the invention, there is provided a peptide mixture suitable for eliciting an immune response comprising a first and a second peptide, each corresponding to a fragment of the RAS protein wherein:
the first peptide comprises a region of at least 8 amino acids which includes a first mutated position,
the second peptide comprises a region of at least 8 amino acids which includes a second mutated position,
each of said regions of the first and second peptides independently has at least 6 amino acid residues, other than at said first and second mutated positions, which are identical to the corresponding region of the RAS protein,
each of the first and second peptides has a point mutation at said first and second mutated positions,
wherein the first mutated position is position 117 or 146 of the RAS protein and the second mutated position is position 12, 13, 61, 117 or 146 of the RAS protein,
and wherein the point mutation of the first mutated position is different from the point mutation of the second mutated position.

Advantageously, the point mutation of the first peptide is selected from a K117N, a A146T or a A146V mutation.

Conveniently, the point mutation of the second peptide is selected from a K117N, a A146T, a A146V, a G13A, G13C, G13D, G13R, G13S, a G13V, G12A, G12C, G12D, G12R, G12S, a G12V, Q61E, Q61H, Q61K, Q61L, a Q61P or a Q61R mutation.

Preferably, the first peptide has a point mutation at position 146 of the RAS protein, and the second peptide has a mutation at position 61 of the RAS peptide.

Advantageously, the peptide mixture comprises at least one further peptide corresponding to a fragment of the RAS protein, wherein:
said at least one further peptide comprises a region of at least 8 amino acids which includes a mutated position,
said region of said at least one further peptide has at least 6 amino acid residues, other than at said mutated position, which are identical to the corresponding region of the RAS protein,
said at least one further peptide has a point mutation at said mutated position,
wherein said mutated position of the at least one further peptide is position 12, 13, 61, 117 or 146 of the RAS protein, and
the point mutation of said at least one further peptide is different from the point mutation of each of the first and second RAS peptides.

Preferably, the first mutated position is position 146 of the RAS protein and the second mutated position is position 12, 13 or 61 of the RAS protein.

Advantageously, the first mutated position is position 146 of the RAS protein, the second mutated position is position 12 of the RAS protein and the mutated position of the at least one further peptide is position 13 of the RAS protein.

Conveniently, the first mutated position is position 146 of the RAS protein, the second mutated position is position 13 of the RAS protein, and the mutated position of the at least one further peptide is position 61 of the RAS protein.

Preferably, the first mutated position is position 146 of the RAS protein, the second mutated position is position 12 of the RAS protein, the first further peptide has a mutated position which is position 13 of the RAS protein and the peptide mixture further comprises a second further peptide having a mutated position which is position 61 of the RAS protein.

Advantageously, the first mutated position is position 146 of the RAS protein, the second mutated position is position 13 of the RAS protein, the first further peptide has a mutated position which is position 13 of the RAS protein, and the peptide mixture further comprises a second further peptide having a mutated position which is position 61 of the RAS protein.

Conveniently, the peptide mixture comprises a first peptide having a A146T mutation, a second peptide having a G13R mutation, a third peptide having a G13V mutation, a fourth peptide having a Q61R mutation, a fifth peptide having a Q61K mutation, a sixth peptide having a Q61H mutation and a seventh peptide having a Q61L mutation.

Preferably, the peptide mixture comprises or consists essentially of a first peptide comprising the sequence represented by SEQ ID NO: 1 or 2, a second peptide comprising the sequence represented by SEQ ID NO: 33, a third peptide comprising the sequence represented by SEQ ID NO: 34, a fourth peptide comprising the sequence represented by SEQ ID NO: 35, a fifth peptide comprising the sequence represented by SEQ ID NO: 36, a sixth peptide comprising the sequence represented by SEQ ID NO: 37 and a seventh peptide comprising the sequence represented by SEQ ID NO: 38.

In a third aspect of the invention, there is provided a T-cell specific for the peptide according to the first or second aspect described above, when presented on an MHC molecule.

In a fourth aspect of the invention, there is provided a T-cell preparation comprising a T-cell according to the third aspect described above.

In a fifth aspect of the present invention, there is provided a T-cell mixture comprising T-cells specific for each of the peptides in one of the peptide mixtures according to the second aspect described above, when presented on an MHC cell.

In a sixth aspect of the present invention, there is provided a T-cell receptor, or an antigen-binding fragment thereof, specific for a peptide according to the first aspect described above or for a peptide for use according to In a sixth aspect of the present invention, there is provided a, when presented on an MHC molecule.

In a seventh aspect of the present invention, there is provided a nucleic acid which comprises a nucleotide sequence which encodes the peptide according to the first aspect described above, a peptide for use according to the first aspect described above or a T-cell receptor according to the sixth aspect described above.

In an eighth aspect of the present invention, there is provided a vector comprising a nucleic acid according to the seventh aspect described above.

In a ninth aspect of the present invention, there is provided a host cell comprising a vector according to the eighth aspect described above.

In a tenth aspect of the present invention, there is provided a pharmaceutical composition comprising a peptide according to the first aspect described above, a peptide for use according to the first aspect described above, a peptide mixture according to the second aspect described above, a T-cell according to the third aspect described above, a T-cell preparation according to the fourth aspect described above, a T-cell mixture according to the fifth aspect described above, a T-cell receptor or antigen-binding fragment thereof according to the sixth aspect described above, a nucleic acid according to the seventh aspect described above, a vector according to the eighth aspect described above or a host cell according to the ninth aspect described above, and a pharmaceutically acceptable carrier, diluent and/or excipient.

In an eleventh aspect of the present invention, there is provided a peptide according to the first aspect described above, a peptide for use according to the first aspect described above, a peptide mixture according to the second aspect described above, a T-cell according to the third aspect described above, a T-cell preparation according to the fourth aspect described above, a T-cell mixture according to the fifth aspect described above, a T-cell receptor or antigen-binding fragment thereof according to the sixth aspect described above, a nucleic acid according to the seventh aspect described above, a vector according to the eighth aspect described above, a host cell according to the ninth aspect described above, or a pharmaceutical composition according to the tenth aspect described above, for use in the prophylaxis and/or treatment of cancer.

Advantageously, the cancer is adrenal gland, autonomic ganglia, biliary tract, bone, breast, central nervous system, cervical, colorectal, endometrial, haematopoietic, lymphoid, kidney, large intestine, liver, lung, oesophagus, ovarian, pancreatic, prostate, salivary gland, skin, small intestine, stomach, testicular, thymus, thyroid, upper aerodigestive tract and urinary tract cancer and/or malignant melanoma.

Preferably, the cancer is colorectal cancer.

In a twelfth aspect of the invention, there is provided a method of treatment or prophylaxis of cancer, wherein said method comprises administering a peptide according to the first aspect described above, a peptide for use according to the first aspect described above, a peptide mixture according to the second aspect described above, a T-cell according to the third aspect described above, a T-cell preparation according to the fourth aspect described above, a T-cell mixture according to the fifth aspect described above, a T-cell receptor or an antigen-binding fragment thereof according to the sixth aspect described above, a nucleic acid according to the seventh aspect described above, a vector according to the eighth aspect described above, or a pharmaceutical composition according to the ninth aspect described above to a subject in need thereof.

In a thirteenth aspect of the invention, there is provided a method of treatment or prophylaxis of cancer, wherein said method comprises:
a) identifying RAS protein mutations in a sample taken from a patient,
b) selecting a peptide according to the first aspect of the invention comprising a point mutation corresponding to at least one of the RAS protein mutations identified in the sample; a peptide for use according to the first aspect of the invention comprising a point mutation corresponding to at least one of the RAS protein mutations identified in the sample; a peptide mixture according to the second aspect of the invention comprising a peptide comprising a point mutation corresponding to at least one of the RAS protein mutations identified in the sample; a T-cell according to the third aspect of the invention specific for a peptide, when presented on an MHC molecule, comprising a point mutation corresponding to at least one of the RAS mutations identified in the sample; a T-cell preparation according to the fourth aspect of the invention comprising T-cells specific for a peptide, when presented on an MHC molecule, comprising a point mutation corresponding to at least one of the RAS protein mutations identified in the sample; a T-cell mixture according to the fifth aspect of the invention comprising T-cells specific for a peptide, when presented on an MHC molecule, comprising a point mutation corresponding to at least one of the RAS protein mutations identified in the sample; a T-cell receptor or an antigen-binding fragment thereof according to the sixth aspect of the invention specific for a peptide comprising a point mutation corresponding to at least one of the RAS protein mutations identified in the sample; a nucleic acid according to the seventh aspect of the invention, encoding a peptide comprising a point mutation corresponding to at least one of the RAS protein mutations identified in the sample; a vector according to the eighth aspect of the invention, encoding a peptide comprising a point mutation corresponding to at least one of the RAS protein mutations identified in the sample; or a pharmaceutical composition according to the ninth aspect of the invention, comprising a peptide, a peptide for use as a vaccine or medicament, a peptide mixture comprising a point mutation corresponding to at least one of the RAS protein mutations identified in the sample, or a T-cell, a T-cell preparation or a T-cell mixture comprising T-cells specific for a peptide, when presented on an MHC molecule, comprising a point mutation corresponding to at least one of the RAS protein mutations identified in the sample, or a T-cell receptor or an antigen-binding fragment thereof specific for a peptide comprising a point mutation corresponding to at least one of the RAS protein mutations identified in the sample, or a nucleic acid or a vector encoding a peptide comprising a point mutation corresponding to at least one of the RAS protein mutations identified in the sample, and
c) administering the peptide, peptide mixture, T-cell, T-cell preparation, T-cell mixture, antibody or antigen-binding fragment thereof, nucleic acid or vector to the patient.

The term “peptide” as used herein, refers to a polymer of amino acid residues that is (or has a sequence that corresponds to) a fragment of a longer protein. The term also applies to amino acid polymers in which one or more amino acid residues is a modified residue, or a non-naturally occurring residue, such as an artificial chemical mimetic of a corresponding naturally-occurring amino acid, as well as to naturally occurring amino acid polymers.

The term “amino acid” as used herein refers to naturally occurring and synthetic amino acids, as well as amino acid analogues and amino acid mimetics that have a function that is similar to the naturally occurring amino acids. Naturally occurring amino acids are those encoded by the genetic code, as well as those modified after translation in cells (e.g. hydroxyproline, gamma-carboxyglutamate, and O-phosphoserine). The phrase “amino acid analogue” refers to compounds that have the same basic chemical structure (an alpha carbon bound to a hydrogen, a carboxy group, an amino group, and an R group) as a naturally occurring amino acid but have a modified R group or modified backbones (e.g. homoserine, norleucine, methionine sulfoxide, methionine methyl sulphonium). The phrase “amino acid mimetic” refers to chemical compounds that have different structures.

The percentage “identity” between two sequences may be determined using the BLASTP algorithm version 2.2.2 (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

The term “immune response”, as used herein, refers in some embodiments to a T-cell-mediated immune response upon presentation of a peptide by major histocompatibility (MHC) molecules on the surface of cells, and in particular refers to activation of T-cells upon presentation of peptide.

The term “RAS protein”, as used herein, refers to the class of small GTPase proteins encoded by the ras proto-oncogene and includes all three isoforms of the RAS protein: HRAS, KRAS and NRAS. In some embodiments, the term “RAS protein” refers to the protein corresponding to UniProtKB/Swiss-Prot accession number P01112.1 and as shown in SEQ ID NO: 39, the protein corresponding to UniProtKB/Swiss-Prot accession number P01116 and as shown in SEQ ID NO: 44, or the protein corresponding to UniProtKB/Swiss-Prot accession number P01111 and as shown in SEQ ID NO: 45.

The term “position 117 of the RAS protein”, as used herein, refers to the one hundred and seventeenth amino acid in the amino acid chain forming the primary structure of the wild-type RAS protein, counting from the N-terminal.

The term “position 146 of the RAS protein”, as used herein, refers to the one hundred and forty-sixth amino acid in the amino acid chain forming the primary structure of the wild-type RAS protein, counting from the N-terminal.

The term “position 13 of the RAS protein”, as used herein, refers to the thirteenth amino acid in the amino acid chain forming the primary structure of the wild-type RAS protein, counting from the N-terminal.

The term “position 12 of the RAS protein”, as used herein, refers to the twelfth amino acid in the amino acid chain forming the primary structure of the wild-type RAS protein, counting from the N-terminal.

The term “position 61 of the RAS protein”, as used herein, refers to the sixty-first amino acid in the amino acid chain forming the primary structure of the wild-type RAS protein, counting from the N-terminal.

The term “the amino acid corresponding to position 117”, as used herein, means an amino acid in a peptide of a RAS protein located in the peptide's amino acid chain at a position corresponding to the one hundred and seventeenth amino acid of the amino acid sequence of the RAS protein, counting from the N-terminal. Corresponding meanings are attributed to the terms “the amino acid corresponding to position 12”, “the amino acid corresponding to position 13”, “the amino acid corresponding to position 61” and “the amino acid corresponding to position 146”.

The term “peptide mixture”, as used herein, refers to two or more peptides which are mixed but not chemically combined. The mixtures may be present as a dry powder, solution, suspension or colloid, and may be homogeneous or heterogeneous.

The term “RAS protein mutations”, as used herein, refers to one or more point mutations present in the RAS proteins present in a sample taken from a subject.

The term “point mutation”, as used herein, refers to the replacement of a single amino acid residue in the polypeptide chain of a protein product with a different amino acid residue.

The term, for example, “a G12V mutation”, as used herein, refers to a point mutation which has resulted in the glycine (G) at position 12 of the wild-type RAS protein being replaced with valine (V). Similar definitions apply to similar terms, such as K117N, A146T, G13C, G13R, Q61H etc.

The term “nucleic acid” or “nucleic acid molecule”, as used herein, refers to a polymer of multiple nucleotides. The nucleic acid may comprise naturally occurring nucleotides or may comprise artificial nucleotides such as peptide nucleotides, morpholin and locked nucleotides as well as glycol nucleotides and threose nucleotides.

The term “nucleotide”, as used herein, refers to naturally occurring nucleotides and synthetic nucleotide analogues that are recognised by cellular enzymes.

The term “pharmaceutical composition”, as used herein, means a pharmaceutical preparation suitable for administration to an intended human or animal subject for therapeutic purposes.

### BRIEF DESCRIPTION OF THE FIGURES

FIG. 1 is a schematic showing the incidence of mutations in the RAS protein in colorectal cancer.

FIG. 2 is a graph showing the incidence of RAS mutations present in all cancers.

FIG. 3 is a graph showing the respective incidence of RAS mutations in all cancers corresponding to peptides in two peptide mixtures (TG02 and TG03) of the present invention.

FIG. 4 is a graph showing the respective incidence of RAS mutations in lung cancer corresponding to peptides in two peptide mixtures (TG02 and TG03) of the present invention.

FIG. 5 is a graph showing the respective incidence of RAS mutations in colorectal cancer corresponding to peptides in two peptide mixtures (TG02 and TG03) of the present invention.

FIG. 6 is a graph showing the respective incidence of RAS mutations in malignant melanoma corresponding to peptides in two peptide mixtures (TG02 and TG03) of the present invention.

FIG. 7 is a graph showing the proliferative T-cell response to three rounds of in vitro stimulation with a mixture of a RAS peptide having a G13C mutation (SEQ ID NO: 19) and a RAS peptide having a G13R mutation (SEQ ID NO: 27), in healthy donors. APC: antigen presenting cells (PBMC), CPM: counts per minute.

FIG. 8 is a graph showing the proliferative T-cell response to three rounds of in vitro stimulation with a RAS peptide mixture consisting of a G13C peptide (SEQ ID NO: 19), a G13D peptide (SEQ ID NO: 20), a G12A peptide (SEQ ID NO: 21), a G12C peptide (SEQ ID NO: 22), a G12D peptide (SEQ ID NO: 23), a G12R peptide (SEQ ID NO: 24), a G12S peptide (SEQ ID NO: 25), a G12V peptide (SEQ ID NO: 26) and a G13R peptide (SEQ ID NO: 27), in a healthy donor. APC: antigen presenting cells (PBMC), CPM: counts per minute.

FIG. 9 is a graph showing the T-cell response to G13C RAS peptide (SEQ ID NO: 19) after three rounds of in vitro stimulation with TG02+13R, in a healthy donor. APC: antigen presenting cells (PBMC), CPM: counts per minute.

FIG. 10 is a graph showing the proliferative response of splenocytes harvested from mice vaccinated with a RAS peptide mixture (TG02; mouse #4 and #7) and TG02+Viscogel™ (mouse #24, #29 and #30). ConA: Concanavalin A (positive control).

FIG. 11 is a graph showing the T-cell proliferation after stimulation of buffy coats from healthy donor (buffy 5) with a A146T peptide (SEQ ID NO:1).

FIG. 12 is a graph showing the peptide-specific T-cell proliferation after stimulation of buffy coat (buffy 5) from a healthy donor with a peptide cocktail consisting of A146T peptide (SEQ ID NO: 1), a G13C peptide (SEQ ID NO: 25), a G13D peptide (SEQ ID NO: 26) and a Q61R peptide (SEQ ID NO: 35).

### BRIEF DESCRIPTION OF THE SEQUENCE LISTING

SEQ ID NO.: 1 shows an amino acid sequence of the KRAS peptide having a A146T mutation.

SEQ ID NO.: 2 shows an amino acid sequence of the NRAS peptide having a A146T mutation.

SEQ ID NO.: 3 shows an amino acid sequence of the KRAS peptide having a A146V mutation.

SEQ ID NO.: 4 shows an amino acid sequence of the NRAS peptide having a A146V mutation.

SEQ ID NO.: 5 shows an amino acid sequence of the KRAS peptide having a K117N mutation.

SEQ ID NO.: 6 shows an amino acid sequence of the NRAS peptide having a K117N mutation.

SEQ ID NO.: 7 shows an amino acid sequence of the RAS peptide having a G13C mutation.

SEQ ID NO.: 8 shows an amino acid sequence of the RAS peptide having a G13R mutation.

SEQ ID NO.: 9 shows an amino acid sequence of the RAS peptide having a G13D mutation.

SEQ ID NO.: 10 shows an amino acid sequence of the RAS peptide having a G13V mutation.

SEQ ID NO.: 11 shows an amino acid sequence of the RAS peptide having a G13A mutation.

SEQ ID NO.: 12 shows an amino acid sequence of the RAS peptide having a G13S mutation.

SEQ ID NO.: 13 shows an amino acid sequence of the RAS peptide having a G12A mutation.

SEQ ID NO.: 14 shows an amino acid sequence of the RAS peptide having a G12C mutation.

SEQ ID NO.: 15 shows an amino acid sequence of the RAS peptide having a G12D mutation.

SEQ ID NO.: 16 shows an amino acid sequence of the RAS peptide having a G12R mutation.

SEQ ID NO.: 17 shows an amino acid sequence of the RAS peptide having a G12S mutation.

SEQ ID NO.: 18 shows an amino acid sequence of the RAS peptide having a G12V mutation.

SEQ ID NO.: 19 shows an amino acid sequence of the RAS peptide having a Q61R mutation.

SEQ ID NO.: 20 shows an amino acid sequence of the RAS peptide having a Q61K mutation.

SEQ ID NO.: 21 shows an amino acid sequence of the RAS peptide having a Q61H mutation.

SEQ ID NO.: 22 shows an amino acid sequence of the RAS peptide having a Q61L mutation.

SEQ ID NO.: 23 shows an amino acid sequence of the RAS peptide having a Q61E mutation.

SEQ ID NO.: 24 shows an amino acid sequence of the RAS peptide having a Q61P mutation.

SEQ ID NO.: 25 shows an amino acid sequence of the RAS peptide of one embodiment of the peptide mixture having a G13C mutation.

SEQ ID NO.: 26 shows an amino acid sequence of the RAS peptide of one embodiment of the peptide mixture having a G13D mutation.

SEQ ID NO.: 27 shows an amino acid sequence of the RAS peptide of one embodiment of the peptide mixture having a G12A mutation.

SEQ ID NO.: 28 shows an amino acid sequence of the RAS peptide of one embodiment of the peptide mixture having a G12C mutation.

SEQ ID NO.: 29 shows an amino acid sequence of the RAS peptide of one embodiment of the peptide mixture having a G12D mutation.

SEQ ID NO.: 30 shows an amino acid sequence of the RAS peptide of one embodiment of the peptide mixture having a G12R mutation.

SEQ ID NO.: 31 shows an amino acid sequence of the RAS peptide of one embodiment of the peptide mixture having a G12S mutation.

SEQ ID NO.: 32 shows an amino acid sequence of the RAS peptide of one embodiment of the peptide mixture having a G12V mutation.

SEQ ID NO.: 33 shows an amino acid sequence of the RAS peptide of one embodiment of the peptide mixture having a G13R mutation.

SEQ ID NO.: 34 shows an amino acid sequence of the RAS peptide of one embodiment of the peptide mixture having a G13V mutation.

SEQ ID NO.: 35 shows the amino acid sequence of the RAS peptide of one embodiment of the peptide mixture having a Q61R mutation.

SEQ ID NO.: 36 shows the amino acid sequence of the RAS peptide of one embodiment of the peptide mixture having a Q61K mutation.

SEQ ID NO.: 37 shows the amino acid sequence of the RAS peptide of one embodiment of the peptide mixture having a Q61H mutation.

SEQ ID NO.: 38 shows the amino acid sequence of the RAS peptide of one embodiment of the peptide mixture having a Q61L mutation.

SEQ ID NO.: 39 shows the full length amino acid sequence of the wild-type HRAS protein.

SEQ ID NO.: 40 shows the amino acid sequence of the wild-type KRAS peptide including position 117 of the RAS protein.

SEQ ID NO.: 41 shows the amino acid sequence of the wild-type NRAS peptide including position 117 of the RAS protein.

SEQ ID NO.: 42 shows the amino acid sequence of the wild-type KRAS peptide including position 146 of the RAS protein.

SEQ ID NO.: 43 shows the amino acid sequence of the wild-type NRAS peptide including position 146 of the RAS protein.

SEQ ID NO: 44 shows the full length amino acid sequence of the wild-type KRAS protein.

SEQ ID NO: 45 shows the full length amino acid sequence of the wild-type NRAS protein.

### DETAILED DESCRIPTION OF THE INVENTION

The invention relates, in general terms, to a peptide of the RAS protein comprising a region which corresponds to a fragment of the RAS protein, wherein the region comprises at least 8 amino acids which includes a mutated position, wherein the peptide has a point mutation at the mutated position, and the mutated position is position 117 or 146 of the RAS protein. As discussed above, these peptides can be used as vaccines against and/or treatments for cancers, and it has now been shown, for the first time, that these mutated peptides are immunogenic. FIG. 11, for example, shows that a peptide having a mutation at position 146 of the RAS protein is immunogenic and stimulates the induction of T-cells.

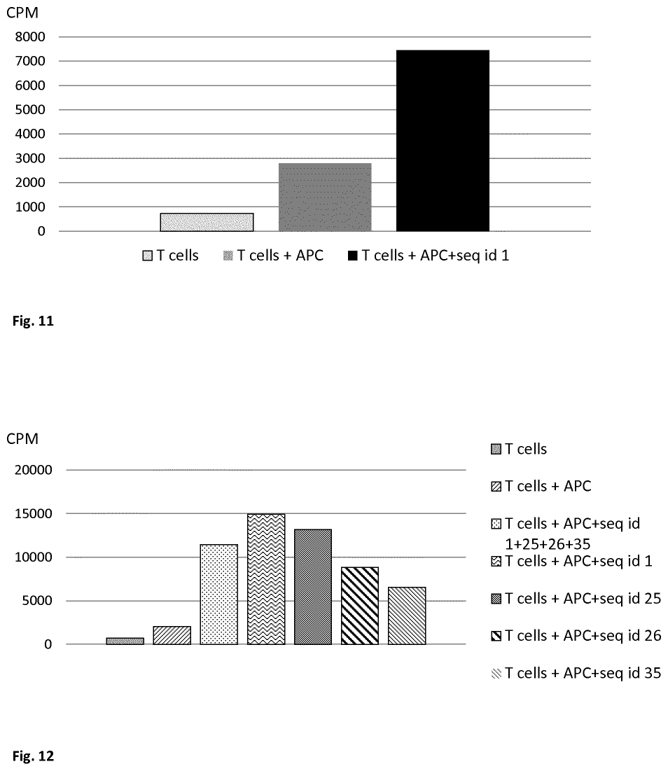

Further, the invention relates to peptide mixtures comprising at least first and second peptides of the RAS protein comprising a region which corresponds to a fragment of the RAS protein, wherein the region comprises at least 8 amino acids, wherein the first peptide includes a first mutated position and the second peptide includes a second mutated position, wherein each of the first and second peptides has a point mutation at the first and second mutated positions, wherein the first mutated position is position 117 or 146 of the RAS protein and the second mutated position is position 12, 13, 61, 117 or 146 of the RAS protein, and wherein each of the mutations is different from each other. FIG. 12 shows that one example of such a peptide mixture is effective in inducing a T-cell response, not only to the peptide mixture, but also to the individual peptides in the peptide mixture, and this is the first time that it has been shown that a peptide mixture comprising peptides having position 146, 13 and 61 mutations are immunogenic when used in combination.

The peptides of the invention may be peptides corresponding to any of HRAS, KRAS or NRAS. All three of these RAS isoforms share sequence identity in all of the regions of exons 2 and 3 responsible for GDP/GTP binding, and share very high sequence homology in all of the regions of exon 4 responsible for GDP/GTP binding, i.e. the regions subject to mutation in cancer. For example, in the region of 25 amino acid sequences centred on position 146 of the RAS protein, both wild-type HRAS and wild-type NRAS have 92% sequence homology (i.e. a difference of two amino acids) with wild-type KRAS. In the region of 25 amino acids centred on position 117 of the RAS protein, wild-type NRAS has 92% homology with wild-type KRAS (i.e. a difference of two amino acids) and wild-type HRAS has 80% homology (i.e. a difference of five amino acids) with wild-type KRAS. The differences in the sequences coded by wild-type exon 4 do not interfere with GDP/GTP binding, and it is the oncogenic mutations specified herein which cause an impact on GDP/GTP binding.

In some embodiments, the peptide comprises at least 8, at least 9, at least 10, at least 12, at least 16, at least 17, at least 18, at least 20, at least 24 or at least 30 amino acids. In preferred embodiments, the peptide comprises at least 8 amino acids. In other preferred embodiments, the peptide comprises at least 17 amino acids.

In some embodiments, the peptide comprises no more than 30 amino acid residues. For example, the peptide comprises no more than 28, 26, 24, 22, 20, 18, 16, 14, 12, 10 or 8 amino acids in certain embodiments.

In some embodiments, the peptide is presented as part of a longer polypeptide comprising elements unrelated to the RAS protein. In particular, the longer polypeptide may comprise one or more regions which are unrelated to the RAS protein.

The peptide has a point mutation at position 117 or 146 of the RAS protein. The wild-type RAS protein comprises lysine (K) at position 117 and alanine (A) at position 146. Thus the mutation at position 117 may be a point mutation from lysine to any other amino acid and the mutation at position 146 may be a point mutation from alanine to any other amino acid. However, K117N, A146T and A146V mutations have been found to be particularly associated with cancer. Thus, in preferred embodiments, the point mutation of the peptide is one of a K117N, A146T and A146V mutation. In particularly preferred embodiments, the point mutation is a A146T or a A146V mutation. In even more preferred embodiments, the point mutation is a A146T mutation, and FIG. 11 shows that such a peptide is immunogenic and is efficient in inducing T-cell proliferation.

In some embodiments, the peptide has at least 20%, at least 25%, at least 30%, at least 37%, at least 50%, at least 60%, at least 66%, at least 75%, at least 85%, at least 95%, at least 99% or 100% sequence identity at positions other than the region including the mutated position. In some embodiments, the peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-6. In preferred embodiments, the peptide has at least 95% sequence identity at positions other than the region including the mutated position to one of SEQ ID NOs: 1-6. In other embodiments, the peptide has 100% sequence identity at positions other than the region including the mutated position to one of SEQ ID NOs: 1-6. In particularly preferred embodiments, the peptide has 100% sequence identity at positions other than the region including the mutated position to one of SEQ ID NOs: 1-4. In even more preferred embodiments, the peptide has 100% sequence identity at positions other than the region including the mutated position to one of SEQ ID NOs: 1 and 2.

In some embodiments of the peptide mixtures mentioned above, each of the first and second peptides independently comprises at least 8, at least 9, at least 10, at least 12, at least 16, at least 17, at least 18, at least 20, at least 24 or at least 30 amino acids. In preferred embodiments, each of the first and second peptides comprises at least 8 amino acids. In other preferred embodiments, each of the first and second peptides comprises at least 17 amino acids. In some embodiments, each of the first and second peptides independently comprises no more than 30 amino acid residues. For example, each of the first and second peptides comprises no more than 28, 26, 24, 22, 20, 18, 16, 14, 12, 10 or 8 amino acids in certain embodiments. The first and the second peptides may have the same number of amino acids or they may have a different number of amino acids.

In some embodiments, each of the first and second peptides has at least 20%, at least 25%, at least 30%, at least 37%, at least 50%, at least 60%, at least 66%, at least 75%, at least 85%, at least 95%, at least 99% or 100% sequence identity at positions other than the region including the mutated position with the RAS protein. In some embodiments, the first peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-6. In preferred embodiments, the first peptide has at least 95% sequence identity at positions other than the region including the mutated position to one of SEQ ID NOs: 1-6. In other embodiments, the first peptide has 100% sequence identity at positions other than the region including the mutated position to one of SEQ ID NOs: 1-6. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-24. In preferred embodiments, the second peptide has at least 95% sequence identity at positions other than the region including the mutated position to one of SEQ ID NOs: 1-24. In other embodiments, the second peptide has 100% sequence identity at positions other than the region including the mutated position to one of SEQ ID NOs: 1-24.

In some embodiments, the first peptide has a percentage sequence identity at positions other than the region including the mutated position to one of SEQ ID NOs: 1-6 and the second peptide has a different percentage sequence identity at positions other than the region including the mutated position to a different one of SEQ ID NOs: 1-24. In other embodiments, the first peptide has a percentage sequence identity at positions other than the region including the mutated position to one of SEQ ID NOs: 1-6 and the second peptide has the same percentage sequence identity at positions other than the region including the mutated position to one of SEQ ID NOs: 1-24. In all embodiments each of the first and second peptides is capable of eliciting an immune response.

When the mutated position of the first peptide is position 117 of the RAS protein, the point mutation of the first peptide may be a point mutation from lysine (K) to any other amino acid. When the mutated position of the first peptide is position 146 of the RAS protein, the point mutation of the first peptide may be a point mutation from alanine (A) to any other amino acid. In some embodiments, the point mutation of the first peptide is one of a K117N, A146T and A146V mutation. In some embodiments, the point mutation of the first peptide is a A146T mutation, and in other embodiments, the point mutation of the first peptide is a A146V mutation. In further embodiments, the point mutation of the first peptide is a K117N mutation.

The point mutation of the second peptide may be a point mutation from the wild-type amino acid to any other amino acid. For example, the wild-type RAS protein comprises glycine (G) at each of positions 12 and 13, and glutamine (Q) at position 61. Thus, when the mutated position of the second peptide is position 13 of the RAS protein, the mutation at position 13 may be a point mutation from glycine to any other amino acid. Similarly, when the mutated position of the second peptide is position 12 or 61 of the RAS protein, the mutation at position 12 or 61 may be a point mutation from glycine or glutamine, as appropriate, to any other amino acid. When the mutated position of the second peptide is position 117 or 146 of the RAS protein, the mutation at the mutated position may be from lysine or alanine, respectively, to any other amino acid. In preferred embodiments, the point mutation of the second peptide is any one of a K117N, a A146T, a A146V, a G13A, G13C, G13D, G13R, G13S, a G13V, G12A, G12C, G12D, G12R, G12S, a G12V, Q61E, Q61H, Q61K, Q61L, a Q61P or a Q61R mutation, independently of the point mutation of the first peptide.

In some embodiments, the point mutation at the mutated position of the first peptide is a A146T mutation and the point mutation at the mutated position of the second peptide is any one of a A146V, K117N, G13A, G13C, G13D, G13R, G13S, a G13V, G12A, G12C, G12D, G12R, G12S, a G12V, Q61E, Q61H, Q61K, Q61L, a Q61P or a Q61R mutation. In some embodiments, the point mutation of the first peptide is a A146T mutation, and the point mutation of the second peptide is a A146V mutation. In other embodiments, the point mutation of the first peptide is a A146T mutation and the point mutation of the second peptide is a K117N mutation. In other embodiments, the point mutation of the first peptide is a A146T mutation, and the point mutation of the second peptide is a G13C, G13D or a Q61R mutation.

In other embodiments, the point mutation at the mutated position of the first peptide is a A146V mutation and the point mutation at the mutated position of the second peptide is any one of a A146T, K117N, G13A, G13C, G13D, G13R, G13S, a G13V, G12A, G12C, G12D, G12R, G12S, a G12V, Q61E, Q61H, Q61K, Q61L, a Q61P or a Q61R mutation. In some embodiments, the point mutation of the first peptide is a A146V mutation, and the point mutation of the second peptide is a K117N mutation.

In yet other embodiments, the point mutation at the mutated position of the first peptide is a K117N mutation, and the point mutation at the mutated position of the second peptide is any one of a G13A, G13C, G13D, G13R, G13S, a G13V, G12A, G12C, G12D, G12R, G12S, a G12V, Q61E, Q61H, Q61K, Q61L, a Q61P or a Q61R mutation.

In alternative embodiments of the invention, the peptide mixture may comprise at least one further peptide of the RAS protein comprising a region of at least 8 amino acids including a mutated position. The mutated position of the at least one further peptide is position 12, 13, 61, 117 or 146 of the RAS protein, and the at least one further peptide may have a point mutation at the mutated position which is different from the point mutations of the first and second peptides. The point mutation at the mutated position of the at least one further peptide may be any one of a K117N, a A146T, a A146V, a G13A, G13C, G13D, G13R, G13S, a G13V, G12A, G12C, G12D, G12R, G12S, a G12V, Q61E, Q61H, Q61K, Q61L, a Q61P or a Q61R mutation, independently of the point mutations of the first and second peptides. In some embodiments, the mutated position of the at least one further peptide is position 12, 13 or 61 of the RAS protein, preferably a G13A, G13C, G13D, G13R, G13S, a G13V, G12A, G12C, G12D, G12R, G12S, a G12V, Q61E, Q61H, Q61K, Q61L, a Q61P or a Q61R mutation, independently of the point mutations of the first and second peptides. In some embodiments, the mutated position of the at least one further peptide is G13C, G13D or a Q61R mutation, independently of the point mutations of the first and second peptides.

The peptide mixture may comprise more than one further peptide. For example, the peptide mixture may comprise two further peptides, three further peptides, four further peptides, or more. In embodiments in which the peptide mixture comprises one or more further peptides, each of the further peptides independently has the characteristics of the at least one further peptide described herein.

In embodiments where the peptide mixture comprises at least one further peptide, each of the first, second and at least one further peptide independently comprises at least 8, at least 9, at least 10, at least 12, at least 16, at least 17, at least 18, at least 20, at least 24 or at least 30 amino acids. In preferred embodiments, each of the peptides comprises at least 8 amino acids. In other preferred embodiments, each of the peptides comprises at least 17 amino acids. In further embodiments, each of the peptides comprises at least 18 amino acids. In some embodiments, each of the first, second and at least one further peptide independently comprises no more than 30 amino acid residues. For example, each of the first and second peptides comprises no more than 28, 26, 24, 22, 20, 18, 16, 14, 12, 10 or 8 amino acids in certain embodiments. In general, each peptide in the peptide mixture may comprise a different number of amino acids to one or more of the other peptides in the peptide mixture.

In embodiments where the peptide mixture comprises at least one further peptide, the amino acid corresponding to the mutated position of the RAS protein has a point mutation. As with the second peptide, the point mutation at the mutated position may be from the wild-type amino acid to any other amino acid. Thus, when the at least one further peptide comprises position 146 of the RAS protein, the point mutation may be from alanine (A) to any other amino acid. When the at least one further peptide comprises position 117 of the RAS protein, the point mutation may be from lysine (K) to any other amino acid. When the at least one further peptide comprises position 61 of the RAS protein, the point mutation may be from glutamine (Q) to any other amino acid. When the at least one further peptide comprises position 13 of the RAS protein, the point mutation may be from glycine (G) to any other amino acid. When the at least one further peptide comprises position 12 of the RAS protein, the point mutation may be from glycine (G) to any other amino acid.

In embodiments where the mutated position of the at least one further peptide is position 146 of the RAS protein, the peptide may have at least 20%, at least 25%, at least 30%, at least 40%, at least 50%, at least 60%, at least 66%, at least 75%, at least 85%, at least 95%, at least 99% or 100% sequence identity at positions other than the region including position 146 with the RAS protein. In some embodiments, the at least one further peptide has at least 20%, at least 25%, at least 30%, at least 40%, at least 50%, at least 60%, at least 66%, at least 75%, at least 85%, at least 95%, at least 99% or 100% sequence identity at positions other than the region including position 146 to one of SEQ ID NO: 1-4.

In embodiments where the mutated position of the at least one further peptide is position 117 of the RAS protein, the peptide may have at least 20%, at least 25%, at least 30%, at least 40%, at least 50%, at least 60%, at least 66%, at least 75%, at least 85%, at least 95%, at least 99% or 100% sequence identity at positions other than the region including position 117 with the RAS protein. In some embodiments, the at least one further peptide has at least 20%, at least 25%, at least 30%, at least 40%, at least 50%, at least 60%, at least 66%, at least 75%, at least 85%, at least 95%, at least 99% or 100% sequence identity at positions other than the region including position 117 to one of SEQ ID NO: 5 or 6.

In embodiments where the mutated position of the at least one further peptide is position 13 of the RAS protein, the peptide may have at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. In some embodiments, the at least one further peptide has at least 20%, at least 25%, at least 30%, at least 40%, at least 50%, at least 60%, at least 66%, at least 75%, at least 85%, at least 95%, at least 99% or 100% sequence identity at positions other than the region including position 13 to one of SEQ ID NO: 7-12, 25, 26, 33 and 34. In some embodiments, there is more than one further peptide of the RAS protein comprising a region of at least 8 amino acids including position 13 of the RAS protein and having a point mutation at the amino acid corresponding to position 13 of the RAS protein. In such embodiments, each of the peptides having a position 13 mutation has a different point mutation.

In embodiments where the mutated position of the at least one further peptide is position 12 of the RAS protein, the peptide may have at least 20%, at least 25%, at least 30%, at least 40%, at least 50%, at least 60%, at least 66%, at least 75%, at least 85%, at least 95%, at least 99% or 100% sequence identity at positions other than the region including position 12 with the RAS protein. In some embodiments, the at least one further peptide has at least 20%, at least 25%, at least 30%, at least 40%, at least 50%, at least 60%, at least 66%, at least 75%, at least 85%, at least 95%, at least 99% or 100% sequence identity at positions other than the region including position 12 to one of SEQ ID NO: 13-18 and 27-32. In some embodiments, there is more than one further peptide of the RAS protein comprising a region of at least 8 amino acids including position 12 of the RAS protein and having a point mutation at the amino acid corresponding to position 12 of the RAS protein. In such embodiments, each of the peptides having a position 12 mutation has a different point mutation.

In embodiments where the mutated position of the at least one further peptide is position 61 of the RAS protein, the peptide may have at least 20%, at least 25%, at least 30%, at least 40%, at least 50%, at least 60%, at least 66%, at least 75%, at least 85%, at least 95%, at least 99% or 100% sequence identity at positions other than the region including position 61 with the RAS protein. In some embodiments, the at least one further peptide has at least 20%, at least 25%, at least 30%, at least 40%, at least 50%, at least 60%, at least 66%, at least 75%, at least 85%, at least 95%, at least 99% or 100% sequence identity at positions other than the region including position 61 to one of SEQ ID NO: 19-24 and 35-38. In some embodiments, there is more than one further peptide of the RAS protein comprising a region of at least 8 amino acids including position 61 of the RAS protein and having a point mutation at the amino acid corresponding to position 61 of the RAS protein. In such embodiments, each of the peptides having a position 61 mutation has a different point mutation.

In some embodiments, the peptide mixture comprises a first, second and third peptide, wherein the mutated position of the first peptide is position 146 of the RAS protein, the mutated position of the second peptide is position 12, 13 or 61 of the RAS protein, and the mutated position of the third peptide is position 12, 13 or 61 of the RAS protein, wherein the point mutation of the second peptide is different from the point mutation of the third peptide.

In some embodiments, the mutated position of the first peptide is position 146 of the RAS protein, the mutated position of the second peptide is position 12 of the RAS protein and the mutated position of the third peptide is position 13 or position 61 of the RAS protein. In some embodiments, the mutated position of the first peptide is position 146 of the RAS protein, the mutated position of the second peptide is position 12 of the RAS protein and the mutated position of the third peptide is position 13 of the RAS protein, while in other embodiments the mutated position of the first peptide is position 146 of the RAS protein, the mutated position of the second peptide is position 12 of the RAS protein and the mutated position of the third peptide is position 61 of the RAS protein.

In other embodiments, the mutated position of the first peptide is position 146 of the RAS protein, the mutated position of the second peptide is position 13 of the RAS protein, and the mutated position of the third peptide is position 12 or 61 of the RAS protein. In some embodiments, the mutated position of the first peptide is position 146 of the RAS protein, the mutated position of the second peptide is position 13 of the RAS protein, and the mutated position of the third peptide is position 61 of the RAS protein. In other embodiments, the mutated position of the first peptide is position 146 of the RAS protein, the mutated position of the second peptide is position 13 of the RAS protein, and the mutated position of the third peptide is position 13 of the RAS protein.

In other embodiments, the peptide mixture comprises a first, second and third peptide, wherein the point mutation of the first peptide is a A146T mutation, the point mutation of the second peptide is a A146V mutation, and the point mutation of the third peptide is a K117N mutation. The first peptide may have at least 20%, at least 25%, at least 30%, at least 40%, at least 50%, at least 60%, at least 66%, at least 75%, at least 85%, at least 95%, at least 99% or 100% sequence identity at positions other than the region including position 146 with the RAS protein. In some embodiments, the first peptide has at least at least 20%, at least 25%, at least 30%, at least 40%, at least 50%, at least 60%, at least 66%, at least 75%, at least 85%, at least 95%, at least 99% or 100% sequence identity at positions other than the region including position 146 to one of SEQ ID NO: 1 or 2. The second peptide may have at least 20%, at least 25%, at least 30%, at least 40%, at least 50%, at least 60%, at least 66%, at least 75%, at least 85%, at least 95%, at least 99% or 100% sequence identity at positions other than the region including position 146 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 146 to one of SEQ ID NO: 3 or 4. The third peptide may have at least 20%, at least 25%, at least 30%, at least 40%, at least 50%, at least 60%, at least 66%, at least 75%, at least 85%, at least 95%, at least 99% or 100% sequence identity at positions other than the region including position 117 with the RAS protein. In some embodiments, the third peptide has at least 20%, at least 25%, at least 30%, at least 40%, at least 50%, at least 60%, at least 66%, at least 75%, at least 85%, at least 95%, at least 99% or 100% sequence identity at positions other than the region including position 117 to one of SEQ ID NO: 5 or 6. In some embodiments, the peptide mixture consists of a first peptide, a second peptide and a third peptide, wherein the point mutation of the first peptide is a A146T mutation, the point mutation of the second peptide is a A146V mutation, and the point mutation of the third peptide is a K117N mutation, and the peptides are as described above. Any combination of the above-mentioned SEQ ID NOs is envisaged in the peptide mixtures of the present invention.

In some embodiments, the peptide mixture comprises a fourth peptide of the RAS protein comprising a region of at least 8 amino acids including a mutated position of the RAS protein. The mutated position of the fourth peptide is position 12, 13, 61, 117 or 146 of the RAS protein, independently of the mutated position of the first, second and third peptides. The fourth peptide has a point mutation at the mutated position which is different from the point mutations of each of the first, second and third peptides. As with the third peptide, discussed above, the point mutation of the fourth peptide may be from the wild-type amino acid to any other amino acid. In preferred embodiments, the point mutation of the fourth peptide is a K117N, a A146T, a A146V, a G13A, G13C, G13D, G13R, G13S, a G13V, G12A, G12C, G12D, G12R, G12S, a G12V, Q61E, Q61H, Q61K, Q61L, a Q61P or a Q61R mutation.

In some embodiments, the peptide mixture comprises a first, second, third and a fourth peptide as described herein, wherein the mutated position of the first peptide is position 146 of the RAS peptide, the mutated position of the second peptide is position 12 of the RAS protein, the mutated position of the third peptide is position 13 of the RAS protein, and the mutated position of the fourth peptide is position 61 of the RAS protein. Independently of the point mutation of the other peptides, the point mutation of the first peptide may be a A146T or a A146V mutation, the point mutation of the second peptide may be a G12A, G12C, G12D, G12R, G12S, or a G12V mutation, the point mutation of the third peptide may be a G13A, G13C, G13D, G13R, G13S, or a G13V mutation, and the point mutation of the fourth peptide may be a Q61E, Q61H, Q61K, Q61L, a Q61P or a Q61R mutation. The first peptide may have at least 20%, at least 25%, at least 30%, at least 40%, at least 50%, at least 60%, at least 66%, at least 75%, at least 85%, at least 95%, at least 99% or 100% sequence identity at positions other than the region including position 146 with the RAS protein. In some embodiments, the first peptide has at least 20%, at least 25%, at least 30%, at least 40%, at least 50%, at least 60%, at least 66%, at least 75%, at least 85%, at least 95%, at least 99% or 100% sequence identity at positions other than the region including position 146 with one of SEQ ID NOs: 1-4. The second peptide may have at least 20%, at least 25%, at least 30%, at least 40%, at least 50%, at least 60%, at least 66%, at least 75%, at least 85%, at least 95%, at least 99% or 100% sequence identity at positions other than the region including position 12 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 12 with one of SEQ ID NOs: 13-18 and 27-32. The third peptide may have at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. In some embodiments, the third peptide has at least 20%, at least 25%, at least 30%, at least 40%, at least 50%, at least 60%, at least 66%, at least 75%, at least 85%, at least 95%, at least 99% or 100% sequence identity at positions other than the region including position 13 with one of SEQ ID NOs: 7-12, 25, 26, 33 and 34. The fourth peptide may have at least 20%, at least 25%, at least 30%, at least 40%, at least 50%, at least 60%, at least 66%, at least 75%, at least 85%, at least 95%, at least 99% or 100% sequence identity at positions other than the region including position 61 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 61 with one of SEQ ID NOs: 19-24 and 35-38. Any combination of the above-mentioned SEQ ID NOs is envisaged in peptide mixtures of the present invention.

In some embodiments, the peptide mixture comprises a first, second, third and a fourth peptide as described herein, wherein the mutated position of the first peptide is position 146 of the RAS peptide, the mutated position of the second peptide is position 13 of the RAS protein, the mutated position of the third peptide is position 13 of the RAS protein, and the mutated position of the fourth peptide is position 61 of the RAS protein. Independently of the point mutation of the other peptides, the point mutation of the first peptide may be a A146T or a A146V mutation, the point mutation of the second peptide may be a G13A, G13C, G13D, G13R, G13S, or a G13V mutation, the point mutation of the third peptide may be a G13A, G13C, G13D, G13R, G13S, or a G13V mutation, wherein the point mutation of the second peptide is different from the point mutation of the third peptide, and the point mutation of the fourth peptide may be a Q61E, Q61H, Q61K, Q61L, a Q61P or a Q61R mutation. The first peptide may have at least 20%, at least 25%, at least 30%, at least 40%, at least 50%, at least 60%, at least 66%, at least 75%, at least 85%, at least 95%, at least 99% or 100% sequence identity at positions other than the region including position 146 with the RAS protein. In some embodiments, the first peptide has at least 20%, at least 25%, at least 30%, at least 40%, at least 50%, at least 60%, at least 66%, at least 75%, at least 85%, at least 95%, at least 99% or 100% sequence identity at positions other than the region including position 146 with one of SEQ ID NOs: 1-4. The second peptide may have at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 13 with one of SEQ ID NOs: 7-12, 25, 26, 33 and 34. The third peptide may have at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. In some embodiments, the third peptide has at least 20%, at least 25%, at least 30%, at least 40%, at least 50%, at least 60%, at least 66%, at least 75%, at least 85%, at least 95%, at least 99% or 100% sequence identity at positions other than the region including position 13 with one of SEQ ID NOs: 7-12, 25, 26, 33 and 34. The fourth peptide may have at least 20%, at least 25%, at least 30%, at least 40%, at least 50%, at least 60%, at least 66%, at least 75%, at least 85%, at least 95%, at least 99% or 100% sequence identity at positions other than the region including position 61 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 61 with one of SEQ ID NOs: 19-24 and 35-38. Any combination of the above-mentioned SEQ ID NOs is envisaged in peptide mixtures of the present invention.

In some embodiments, the peptide mixture comprises a first, second, third and a fourth peptide as described herein, wherein the mutated position of the first peptide is position 117 of the RAS peptide, the mutated position of the second peptide is position 12 of the RAS protein, the mutated position of the third peptide is position 13 of the RAS protein, and the mutated position of the fourth peptide is position 61 of the RAS protein. Independently of the point mutation of the other peptides, the point mutation of the first peptide may be a K117N mutation, the point mutation of the second peptide may be a G12A, G12C, G12D, G12R, G12S, or a G12V mutation, the point mutation of the third peptide may be a G13A, G13C, G13D, G13R, G13S, or a G13V mutation, and the point mutation of the fourth peptide may be a Q61E, Q61H, Q61K, Q61L, a Q61P or a Q61R mutation. The first peptide may have at least 20%, at least 25%, at least 30%, at least 40%, at least 50%, at least 60%, at least 66%, at least 75%, at least 85%, at least 95%, at least 99% or 100% sequence identity at positions other than the region including position 117 with the RAS protein. In some embodiments, the first peptide has at least 20%, at least 25%, at least 30%, at least 40%, at least 50%, at least 60%, at least 66%, at least 75%, at least 85%, at least 95%, at least 99% or 100% sequence identity at positions other than the region including position 117 with one of SEQ ID NOs: 5 or 6. The second peptide may have at least 20%, at least 25%, at least 30%, at least 40%, at least 50%, at least 60%, at least 66%, at least 75%, at least 85%, at least 95%, at least 99% or 100% sequence identity at positions other than the region including position 12 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 12 with one of SEQ ID NOs: 13-18 and 27-32. The third peptide may have at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. In some embodiments, the third peptide has at least 20%, at least 25%, at least 30%, at least 40%, at least 50%, at least 60%, at least 66%, at least 75%, at least 85%, at least 95%, at least 99% or 100% sequence identity at positions other than the region including position 13 with one of SEQ ID NOs: 7-12, 25, 26, 33 and 34. The fourth peptide may have at least 20%, at least 25%, at least 30%, at least 40%, at least 50%, at least 60%, at least 66%, at least 75%, at least 85%, at least 95%, at least 99% or 100% sequence identity at positions other than the region including position 61 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 61 with one of SEQ ID NOs: 19-24 and 35-38. Any combination of the above-mentioned SEQ ID NOs is envisaged in peptide mixtures of the present invention.

In some embodiments, the peptide mixture comprises a first, second, third, a fourth and a fifth peptide as described herein, wherein the mutated position of the first peptide is position 146 of the RAS peptide, the mutated position of the second peptide is position 12 of the RAS protein, the mutated position of the third peptide is position 13 of the RAS protein, the mutated position of the fourth peptide is position 61 of the RAS protein and the mutated position of the fifth peptide is position 117 of the RAS protein. Independently of the point mutation of the other peptides, the point mutation of the first peptide may be a A146T or a A146V mutation, the point mutation of the second peptide may be a G12A, G12C, G12D, G12R, G12S, or a G12V mutation, the point mutation of the third peptide may be a G13A, G13C, G13D, G13R, G13S, or a G13V mutation, the point mutation of the fourth peptide may be a Q61E, Q61H, Q61K, Q61L, a Q61P or a Q61R mutation and the point mutation of the fifth peptide may be a K117N mutation. The first peptide may have at least 20%, at least 25%, at least 30%, at least 40%, at least 50%, at least 60%, at least 66%, at least 75%, at least 85%, at least 95%, at least 99% or 100% sequence identity at positions other than the region including position 146 with the RAS protein. In some embodiments, the first peptide has at least 20%, at least 25%, at least 30%, at least 40%, at least 50%, at least 60%, at least 66%, at least 75%, at least 85%, at least 95%, at least 99% or 100% sequence identity at positions other than the region including position 146 with one of SEQ ID NOs: 1-4. The second peptide may have at least 20%, at least 25%, at least 30%, at least 40%, at least 50%, at least 60%, at least 66%, at least 75%, at least 85%, at least 95%, at least 99% or 100% sequence identity at positions other than the region including position 12 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 12 with one of SEQ ID NOs: 13-18 and 27-32. The third peptide may have at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. In some embodiments, the third peptide has at least 20%, at least 25%, at least 30%, at least 40%, at least 50%, at least 60%, at least 66%, at least 75%, at least 85%, at least 95%, at least 99% or 100% sequence identity at positions other than the region including position 13 with one of SEQ ID NOs: 7-12, 25, 26, 33 and 34. The fourth peptide may have at least 20%, at least 25%, at least 30%, at least 40%, at least 50%, at least 60%, at least 66%, at least 75%, at least 85%, at least 95%, at least 99% or 100% sequence identity at positions other than the region including position 61 with the RAS protein. In some embodiments, the second peptide has at least 20%, at least 25%, at least 30%, at least 40%, at least 50%, at least 60%, at least 66%, at least 75%, at least 85%, at least 95%, at least 99% or 100% sequence identity at positions other than the region including position 61 with one of SEQ ID NOs: 19-24 and 35-38. The fifth peptide may have at least 20%, at least 25%, at least 30%, at least 40%, at least 50%, at least 60%, at least 66%, at least 75%, at least 85%, at least 95%, at least 99% or 100% sequence identity at positions other than the region including position 117 with the RAS protein. In some embodiments, the fifth peptide has at least 20%, at least 25%, at least 30%, at least 40%, at least 50%, at least 60%, at least 66%, at least 75%, at least 85%, at least 95%, at least 99% or 100% sequence identity at positions other than the region including position 117 with one of SEQ ID NOs: 5 or 6. Any combination of the above-mentioned SEQ ID NOs is envisaged in peptide mixtures of the present invention.

In some embodiments (see Tables 6 and 7), the peptide mixture consists of a peptide having a A146T mutation, a peptide having a G12C mutation, a peptide having a G12D mutation, a peptide having a G12V mutation and a peptide having a G13D mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 1 or 2, and SEQ ID NOs: 9, 14, 15 and 18. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 1 or 2, and SEQ ID NOs: 26, 28, 29 and 32. Tables 6 and 7 show the peptides which are preferably present in these embodiments.

In some embodiments (see Tables 8 and 9), the peptide mixture consists of a peptide having a A146T mutation, a peptide having a Q61R mutation, a peptide having a Q61K mutation, a peptide having a Q61H mutation and a peptide having a Q61L mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 1 or 2, and SEQ ID NOs: 19, 20, 21 and 22. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 1 or 2, and SEQ ID NOs: 35, 36, 37 and 38. Tables 8 and 9 show the peptides which are preferably present in these embodiments.

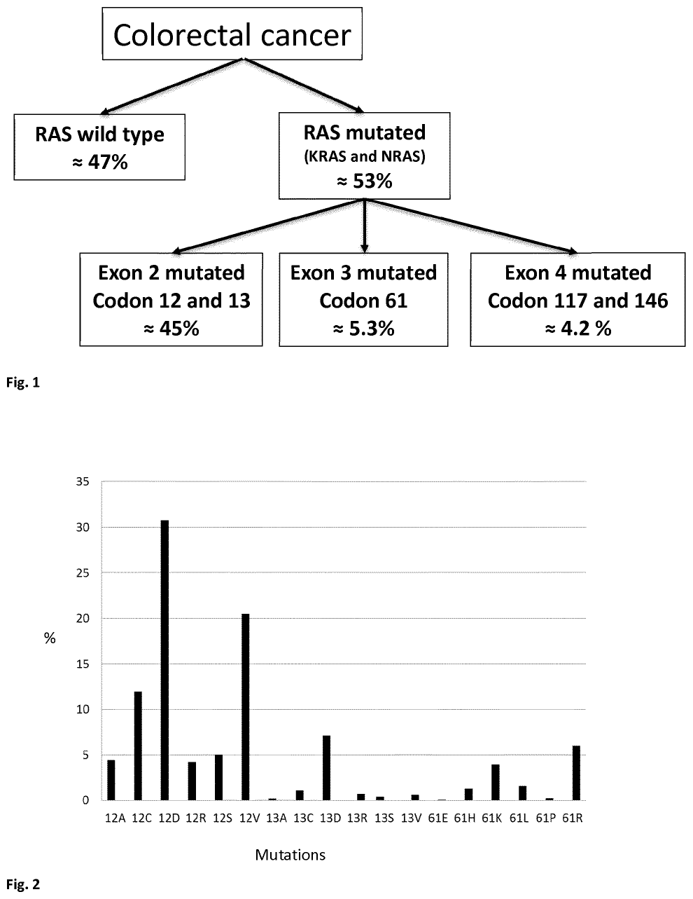

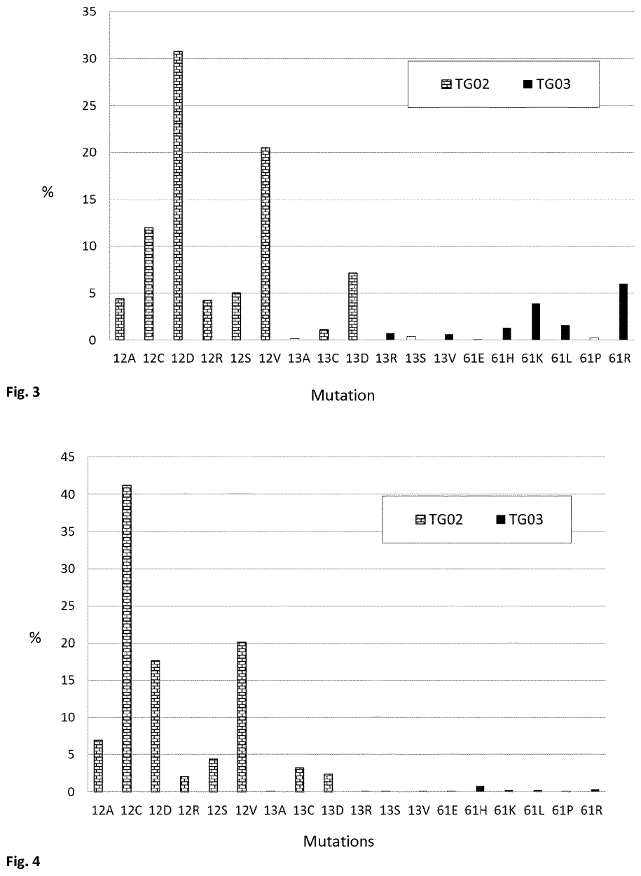

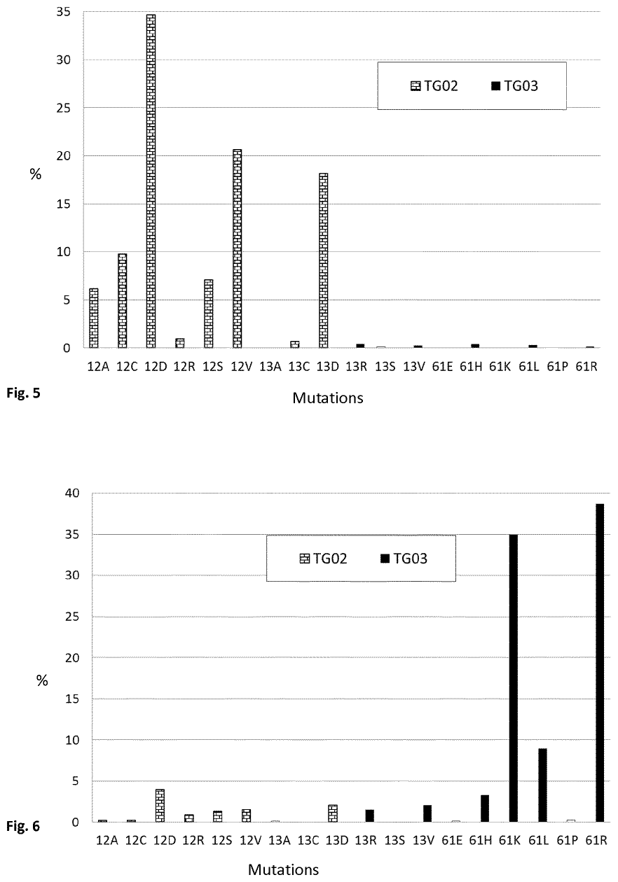

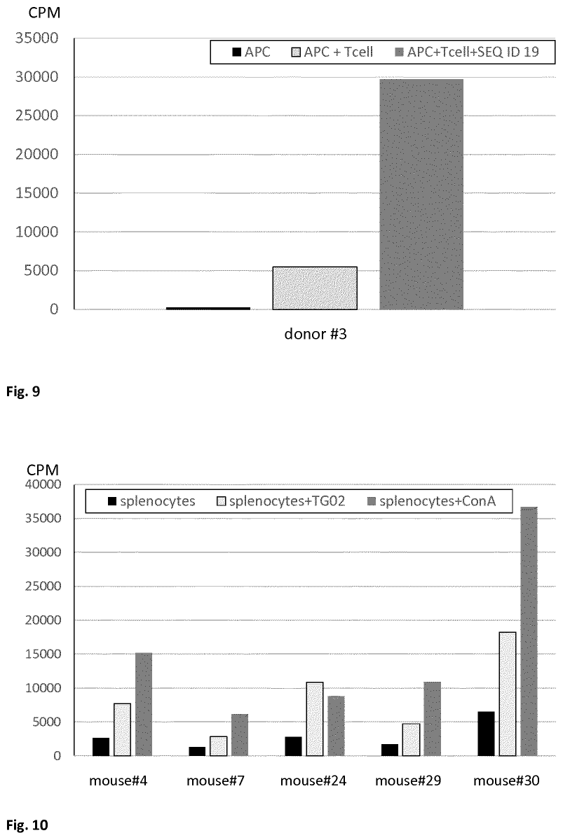

In some embodiments, in addition to a peptide having a point mutation at position 117 and/or a peptide having a point mutation at position 146 of the RAS peptide, the peptide mixture comprises a peptide having a G13C mutation, a peptide having a G13D mutation, a peptide having a G12A mutation, a peptide having a G12C mutation, a peptide having a G12D mutation, a peptide having a G12R mutation, a peptide having a G12S mutation, and a peptide having a G12V mutation, having 100% sequence identity to SEQ ID NOs: 25-32, respectively, and this specific combination of peptides having a mutation at position 12 or 13 of the RAS peptide is referred to herein as TG02. FIG. 3 shows the peptides which are preferably present in TG02. The incidence of these mutations in cancers associated with a RAS mutation, lung cancer and colorectal cancer is shown in FIGS. 2, 4 and 5, respectively. The results of a splenocyte proliferation assay, following vaccination of mice with TG02, are shown in FIG. 9, and show that TG02 is effective in inducing an immune response.

In some embodiments, in addition to a peptide having a point mutation at position 117 and/or a peptide having a point mutation at position 146 of the RAS protein, the peptide mixtures comprises a peptide having a G13R mutation, a peptide having a G13V mutation, a peptide having a Q61R mutation, a peptide having Q61K mutation, a peptide having a Q61H mutation, and a peptide having a Q61L mutation, wherein the peptides have 100% sequence identity to SEQ ID NOs: 33-38, respectively, and this specific combination of peptides having a point mutation at position 13 or 61 of the RAS peptide is referred to herein as TG03. FIG. 3 shows the peptides of TG03. The incidence of these mutations in malignant melanoma is shown in FIG. 6.

In some embodiments (see Tables 10 and 11; referred to herein as TG02+A146T), the peptide mixture consists of a peptide having a A146T mutation, a peptide having a G13C mutation, a peptide having a G13D mutation, a peptide having a G12A mutation, a peptide having a G12C mutation, a peptide having a G12D mutation, a peptide having a G12R mutation, a peptide having a G12S mutation, and a peptide having a G12V mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 1 (KRAS A146T peptide) or SEQ ID NO: 2 (NRAS A146T peptide), and SEQ ID NOs: 7, 9 and 13-18. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 1 (KRAS A146T peptide) or SEQ ID NO: 2 (NRAS A146T peptide), and SEQ ID NOs: 25-32. Tables 10 and 11 show the peptides which are preferably present in this embodiment.

In some embodiments (see Tables 12 and 13; TG02+A146V), the peptide mixture consists of a peptide having a A146V mutation, a peptide having a G13C mutation, a peptide having a G13D mutation, a peptide having a G12A mutation, a peptide having a G12C mutation, a peptide having a G12D mutation, a peptide having a G12R mutation, a peptide having a G12S mutation, and a peptide having a G12V mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NOs: 3 (KRAS A146V peptide) or SEQ ID NO: 4 (NRAS A146V peptide), and SEQ ID NOs: 7, 9 and 13-18. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 3 or 4, and SEQ ID NOs: 25-32. Tables 12 and 13 show the peptides which are preferably present in this embodiment.

In some embodiments (Tables 14 and 15; TG03+A146T), the peptide mixture consists of a peptide having a A146T mutation, a peptide having a G13R mutation, a peptide having a G13V mutation, a peptide having a Q61R mutation, a peptide having Q61K mutation, a peptide having a Q61H mutation, and a peptide having a Q61L mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 1 (KRAS A146T peptide) or SEQ ID NO: 2 (NRAS A146T peptide), and SEQ ID NOs: 8, 10, and 19-22. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 1 (KRAS A146T peptide) or SEQ ID NO: 2 (NRAS A146T peptide), and SEQ ID NOs: 33-38. Tables 14 and 15 show the peptides which are preferably present in this embodiment.

In some embodiments (see Tables 16 and 17; TG03+A146V), the peptide mixture consists of a peptide having a A146V mutation, a peptide having a G13R mutation, a peptide having a G13V mutation, a peptide having a Q61R mutation, a peptide having Q61K mutation, a peptide having a Q61H mutation, and a peptide having a Q61L mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 3 (KRAS A146V peptide) or SEQ ID NO: 4 (NRAS A146V peptide), and SEQ ID NOs: 8, 10, and 19-22. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 3 (KRAS A146V peptide) or SEQ ID NO: 4 (NRAS A146V peptide), and SEQ ID NOs: 33-38. Tables 16 and 17 show the peptides which are preferably present in this embodiment.

In some embodiments (see Tables 18 and 19; TG02+K117N), the peptide mixture consists of a peptide having a K117N mutation, a peptide having a G13C mutation, a peptide having a G13D mutation, a peptide having a G12A mutation, a peptide having a G12C mutation, a peptide having a G12D mutation, a peptide having a G12R mutation, a peptide having a G12S mutation, and a peptide having a G12V mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 5 (KRAS K117N peptide) or SEQ ID NO: 6 (NRAS K117N peptide), and SEQ ID NOs: 7, 9 and 13-18. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 5 (KRAS K117N peptide) or SEQ ID NO: 6 (NRAS K117N peptide), and SEQ ID NOs: 25-32. Tables 18 and 19 show the peptides which are preferably present in this embodiment.

In some embodiments (see Tables 20 and 21; TG03+K117N), the peptide mixture consists of a peptide having a K117N mutation, a peptide having a G13R mutation, a peptide having a G13V mutation, a peptide having a Q61R mutation, a peptide having Q61K mutation, a peptide having a Q61H mutation, and a peptide having a Q61L mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 5 (KRAS K117N peptide) or SEQ ID NO: 6 (NRAS K117N peptide), and SEQ ID NOs: 8, 10, and 19-22. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 5 (KRAS K117N peptide) or SEQ ID NO: 6 (NRAS K117N peptide), and SEQ ID NOs: 33-38. Tables 20 and 21 show the peptides which are preferably present in this embodiment.

In some embodiments (see Tables 22 and 23; TGX3) the peptide mixture consists of a peptide having a A146T mutation, a peptide having a G13C mutation, a peptide having a G13D mutation and a peptide having a Q61R mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position respectively to either SEQ ID NO: 1 (KRAS A146T peptide) or SEQ ID NO: 2 (NRAS A146T peptide), and SEQ ID NOs: 7, 9 and 19. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including the mutated position to either SEQ ID NO: 1 (KRAS A146T peptide) or SEQ ID NO: 2 (NRAS A146T peptide), and SEQ ID NOs: 25, 26 and 35. In some embodiments, the G13C, G13D and Q61R peptides have 100% sequence identity to SEQ ID NOs: 25, 26 and 35, respectively, and the A146T peptide has 100% sequence identity with SEQ ID NO: 1, and this specific combination of peptides is referred to herein as TGX3. Table 22 shows the peptides which are present in TGX3, and FIG. 12 shows that this peptide mixture is effective in inducing a T-cell response to the peptide mixture, as well as to each of the individual peptides in TGX3. Table 23 shows the peptides which are preferably present in an alternative form of TGX3, wherein the peptide having a A146T mutation has 100% sequence identity with SEQ ID NO: 2.

In other embodiments, the peptide mixture consists of a peptide having a A146T, a A146V or a K117N mutation, a peptide having G13C mutation, a peptide having a G13D mutation, a peptide having a G13R mutation, a peptide having a G12A mutation, a peptide having a G12C mutation, a peptide having a G12D mutation, a peptide having a G12R mutation, a peptide having a G12S mutation, and a peptide having a G12V mutation. In such embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including positions 117, 146, 12 or 13 respectively to SEQ ID NOs: 1-9 and 13-18. In some embodiments, the peptide mixture consists of peptides independently having at least 20%, at least 25%, at least 30%, at least 40%, at least 50%, at least 60%, at least 66%, at least 75%, at least 85%, at least 95%, at least 99% or 100% sequence identity at positions other than the region including positions 117, 146, 12 or 13 respectively to SEQ ID NOs: 1-6 and 25-33. The results of a T cell proliferation assay following in vitro stimulation of PMBCs with a peptide mixture consisting of peptides represented by SEQ ID NOs: 25-33 is shown in FIG. 8, and show that T cells were stimulated by this mixture. Thus, RAS peptide mixtures are effective in inducing an immune response.

In some embodiments, the peptide mixture consists of a first, second, third, fourth and fifth peptide as described herein, wherein the mutated position of the first peptide is position 117 or 146 of the RAS protein. The mutated position of each of the second, third, fourth and fifth peptides is position 13 or the RAS protein, and each of the second, third, fourth and fifth peptides independently has at least 20%, at least 25%, at least 30%, at least 40%, at least 50%, at least 60%, at least 66%, at least 75%, at least 85%, at least 95%, at least 99% or 100% sequence identity at positions other than the region including position 13 with the RAS protein. Each of the second, third, fourth and fifth peptides may independently have at least 20%, at least 25%, at least 30%, at least 40%, at least 50%, at least 60%, at least 66%, at least 75%, at least 85%, at least 95%, at least 99% or 100% sequence identity at positions other than the region including position 13 to one of SEQ ID NOs: 7-12, 25, 26, 33 and 34. Each of the second, third, fourth and fifth peptides has a point mutation at the amino acid corresponding to position 13 of the RAS peptide, and each of the second, third, fourth and fifth peptides has a point mutation that is different from the point mutations of the other peptides. In one embodiment, the first peptide is a peptide having a A146T, a A146V or a K117N mutation, the second peptide is a peptide having a G13R mutation, the third peptide is a peptide having a G13A mutation, the fourth peptide is a peptide having a G13S mutation and the fifth peptide is a peptide having a G13V mutation.

Alternative embodiments include a peptide mixture comprising at least six peptides of the RAS protein wherein each of the six peptides comprises a region which corresponds to a fragment of the RAS protein, wherein the region comprises at least 8 amino acids which includes a mutated position, as described herein, and wherein the mutated position of one of the at least six peptides is position 117 or 146 of the RAS protein. Each of the at least six peptides independently has at least 20%, at least 25%, at least 30%, at least 40%, at least 50%, at least 60%, at least 66%, at least 75%, at least 85%, at least 95%, at least 99% or 100% sequence identity at positions other than the region including the mutated position with the RAS protein. In some embodiments, the peptide mixture comprises a first, second, third, fourth, fifth and sixth peptide, wherein the mutated position of the first peptide is position 117 or 146 of the RAS peptide and the mutated position of each of the second, third, fourth, fifth and sixth peptides is position 13 of the RAS protein. The first peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-6 and each of the second, third, fourth, fifth and sixth peptides independently has at least 20%, at least 25%, at least 30%, at least 40%, at least 50%, at least 60%, at least 66%, at least 75%, at least 85%, at least 95%, at least 99% or 100% sequence identity at positions other than the region including position 13 to one of SEQ ID NOs: 7-12, 25, 26, 33 and 34. The first peptide has a point mutation selected from a A146T, a A146V and a K117N mutation, and each of the second, third, fourth, fifth and sixth peptides has a point mutation at the amino acid corresponding to position 13 of the RAS protein, independently selected from a G13A, G13C, G13D, G13R, G13S or a G13V mutation, and the point mutation of each of the second, third, fourth, fifth and sixth peptides is different from the point mutation of the other peptides. In some embodiments, the first peptide has a A146T mutation, In some embodiments the first peptide has a A146V mutation. In other embodiments, the first peptide has a K117N mutation.

In another embodiment, the peptide mixture suitable for eliciting an immune response consists of seven peptides of the RAS protein wherein each peptide comprises a region which corresponds to a fragment of the RAS protein, wherein the region comprises at least 8 amino acids including a mutated position, wherein the mutated position of the first peptide is position 117 or 146 of the RAS protein and the mutated position of each of the second to seventh peptides is position 12 of the RAS protein. The first peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position with the RAS protein and/or has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-6. Each of the second to seventh peptides independently has at least 20%, at least 25%, at least 30%, at least 40%, at least 50%, at least 60%, at least 66%, at least 75%, at least 85%, at least 95%, at least 99% or 100% sequence identity at positions other than the region including position 12 with the RAS protein, and/or independently has at least 20%, at least 25%, at least 30%, at least 40%, at least 50%, at least 60%, at least 66%, at least 75%, at least 85%, at least 95%, at least 99% or 100% sequence identity at positions other than the region including position 12 to one of SEQ ID NOs: 13-18 and 27-32. Each of the second to seventh peptides has a point mutation at the amino acid corresponding to position 12 of the RAS protein, which is selected from a G12A, G12C, G12D, G12R, G12S or a G12V mutation, and the point mutation of each peptide is different from the point mutation of the other peptides.

In another embodiment, the peptide mixture suitable for eliciting an immune response consists of seven peptides of the RAS protein wherein each peptide comprises a region which corresponds to a fragment of the RAS protein, wherein the region comprises at least 8 amino acids including a mutated position, wherein the mutated position of the first peptide is position 117 or 146 of the RAS protein and the mutated position of each of the second to seventh peptides is position 61 of the RAS protein. The first peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position with the RAS protein and/or has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-6. Each of the second to seventh peptides independently has at least 20%, at least 25%, at least 30%, at least 40%, at least 50%, at least 60%, at least 66%, at least 75%, at least 85%, at least 95%, at least 99% or 100% sequence identity at positions other than the region including position 61 with the RAS protein, and/or independently has at least 20%, at least 25%, at least 30%, at least 40%, at least 50%, at least 60%, at least 66%, at least 75%, at least 85%, at least 95%, at least 99% or 100% sequence identity at positions other than the region including position 61 to one of SEQ ID NOs: 19-24 and 35-38. Each of the second to seventh peptides has a point mutation at the amino acid corresponding to position 61 of the RAS protein, which is selected from a Q61E, 61H, Q61K, Q61L, a Q61P or a Q61R mutation, and the point mutation of each peptide is different from the point mutation of the other peptides.

In a further embodiment, a peptide mixture suitable for eliciting an immune response consists of a first, second, third, fourth and fifth peptide of the RAS protein wherein each of the peptides comprises a region which corresponds to a fragment of the RAS protein, wherein the region comprises at least 8 amino acids including a mutated position. The mutated position of the first peptide is position 117 or 146 of the RAS protein, the mutated position of the second, third and fourth peptides is position 12 of the RAS protein, and the mutated position of the fifth peptide is position 13 of the RAS protein. The first peptide has at least 20%, at least 25%, at least 30%, at least 40%, at least 50%, at least 60%, at least 66%, at least 75%, at least 85%, at least 95%, at least 99% or 100% sequence identity at positions other than the region including the mutated position with the RAS protein and/or has at least 20%, at least 25%, at least 30%, at least 40%, at least 50%, at least 60%, at least 66%, at least 75%, at least 85%, at least 95%, at least 99% or 100% sequence identity at positions other than the region including the mutated position to one of SEQ ID NOs: 1-6. Each of the second, third, fourth and fifth peptides has at least 20%, at least 25%, at least 30%, at least 40%, at least 50%, at least 60%, at least 66%, at least 75%, at least 85%, at least 95%, at least 99% or 100% sequence identity at positions other than the region including position 12 or 13 respectively with the RAS protein, and/or independently has at least 20%, at least 25%, at least 30%, at least 40%, at least 50%, at least 60%, at least 66%, at least 75%, at least 85%, at least 95%, at least 99% or 100% sequence identity at positions other than the region including position 12 or 13 respectively to one of SEQ ID NOs: 7-18 and 25-34, respectively. Each of the second, third, fourth and fifth peptides has a point mutation at the amino acid corresponding to said position 12 or 13 of the RAS protein, respectively. In some embodiments, the second peptide is a peptide having a G12A mutation, the third peptide is a peptide having a G12R mutation, the fourth peptide is a peptide having a G12S mutation, and the fifth peptide is a peptide having a G13C mutation.

In general, peptides of the present invention, within a region of 8 amino acids including position 12, 13, 61, 117 or 146, have at least 6 amino acid residues, other than the residue at position 12, 13, 61, 117 or 146 respectively, which are identical to the corresponding region of the RAS protein. Furthermore, in general, peptides of the present invention, at positions other than the region including position 12, 13, 61, 117 or 146 of the RAS protein independently have at least 20%, at least 25%, at least 30%, at least 40%, at least 50%, at least 60%, at least 66%, at least 75%, at least 85%, at least 95%, at least 99% or 100% to one of SEQ ID NOs: 1-38, respectively.

In some embodiments, there is a maximum of 12 different peptides in the peptide mixture. In other embodiments, there is a maximum of 8, 10, 12, 14, or 16 different peptides. In embodiments where the peptide mixture comprises at least one further peptide comprising a region including a mutated position of the RAS protein, the peptide mixture comprises 1, 2, 3, 4, 5 or 6 peptides comprising a region of at least 8 amino acids including a mutated position of the RAS protein and having a point mutation at the mutated position. The mutated position of each of the peptides is, independently, position 117, 146, 12, 13 or 61, and each peptide has a point mutation at the mutated position which is different from the other peptides. In embodiments where the peptide mixture comprises at least one further peptide comprising a region including position 12 of the RAS protein, the peptide mixture comprises 1, 2, 3, 4, 5 or 6 peptides comprising a region of at least 8 amino acids including position 12 of the RAS peptide and having a point mutation at the position corresponding to position 12 of the RAS protein. In embodiments where the peptide mixture comprises at least one further peptide comprising a region of at least 8 amino acids including position 61 of the RAS protein, the peptide mixture comprises 1, 2, 3, 4, 5 or 6 peptides comprising a region including position 61 of the RAS protein and having a point mutation at the amino acid corresponding to position 61 of the RAS protein, wherein each of the peptides has a different point mutation. In embodiments where the peptide mixture comprises at least one further peptide comprising a region of at least 8 amino acids including position 13 of the RAS protein, the peptide mixture comprises 1, 2, 3, 4, 5 or 6 peptides comprising a region including position 13 of the RAS protein and having a point mutation at the amino acid corresponding to position 13 of the RAS protein, wherein each of the peptides has a different point mutation.

In some embodiments, the peptide comprising a region of at least 8 amino acids including position 117 of the RAS peptide comprises positions 107 to 127 of the RAS protein. In alternative embodiments, the peptide comprising a region of at least 8 amino acids including position 117 of the RAS protein comprises positions 108 to 126 of the RAS protein, while in other embodiments the peptide comprises positions 109 to 125 of the RAS protein. In further embodiments, the amino acid corresponding to position 117 of the RAS protein is at the C-terminus of the peptide. In further embodiments, the amino acid corresponding to position 117 of the RAS protein is at the N-terminus of the peptide. In general, the region having at least 8 amino acids including position 117 of the RAS protein may consist of any 8 positions of the RAS protein including position 117. For example, the region having at least 8 amino acids including position 117 may consist of the amino acids from position 110 to position 117, position 111 to position 118, position 112 to position 119, position 113 to position 120, position 114 to position 121, position 115 to position 122, position 116 to position 123 or position 117 to position 124 of the RAS protein. In some embodiments, the amino acid corresponding to position 117 of the RAS protein is in the middle of the peptide.

In some embodiments, the peptide comprising a region of at least 8 amino acids including position 146 of the RAS peptide comprises positions 136 to 156 of the RAS protein. In alternative embodiments, the peptide comprising a region of at least 8 amino acids including position 146 of the RAS protein comprises positions 137 to 155 of the RAS protein, while in other embodiments the peptide comprises positions 138 to 154 of the RAS protein. In further embodiments, the amino acid corresponding to position 146 of the RAS protein is at the C-terminus of the peptide. In further embodiments, the amino acid corresponding to position 146 of the RAS protein is at the N-terminus of the peptide. In general, the region having at least 8 amino acids including position 146 of the RAS protein may consist of any 8 positions of the RAS protein including position 146. For example, the region having at least 8 amino acids including position 146 may consist of the amino acids from position 139 to position 146, position 140 to position 147, position 141 to position 148, position 142 to position 149, position 143 to position 150, position 144 to position 151, position 145 to position 152 or position 146 to position 153 of the RAS protein. In some embodiments, the amino acid corresponding to position 146 of the RAS protein is in the middle of the peptide.

In some embodiments of the peptide mixtures, the peptides comprising a region of at least 8 amino acids including position 12 of the RAS peptide comprise positions 1 to 30 of the RAS protein. In other embodiments, the peptides comprising a region of at least 8 amino acids including position 12 of the RAS protein comprises positions 5 to 21 of the RAS protein. In alternative embodiments, the amino acid corresponding to position 12 of the RAS protein is at the C-terminus of the peptide. In further embodiments, the amino acid corresponding to position 12 of the RAS protein is at the N-terminus of the peptide. In general, the region having at least 8 amino acids including position 12 of the RAS protein may consist of any 8 positions of the RAS protein including position 12. For example, the region having at least 8 amino acids including position 12 may consist of the amino acids from position 5 to position 12, position 6 to position 13, position 7 to position 14, position 8 to position 15, position 9 to position 16, position 10 to position 17, position 11 to position 18 or position 12 to position 19 of the RAS protein.

In some embodiments of the peptide mixtures, the peptides comprising a region of at least 8 amino acids including position 13 of the RAS peptide comprise positions 1 to 30 of the RAS protein. In alternative embodiments, the peptides comprising a region of at least 8 amino acids including position 13 of the RAS protein comprise positions 5 to 21 of the RAS protein. In further embodiments, the amino acid corresponding to position 13 of the RAS protein is at the C-terminus of the peptide. In further embodiments, the amino acid corresponding to position 13 of the RAS protein is at the N-terminus of the peptide. In general, the region having at least 8 amino acids including position 13 of the RAS protein may consist of any 8 positions of the RAS protein including position 13. For example, the region having at least 8 amino acids including position 13 may consist of the amino acids from position 6 to position 13, position 7 to position 14, position 8 to position 15, position 9 to position 16, position 10 to position 17, position 11 to position 18, position 12 to position 19 or position 13 to position 20 of the RAS protein.

In some embodiments of the peptide mixtures, the peptides comprising a region of at least 8 amino acids including position 61 of the RAS peptide comprise positions 47 to 76 of the RAS protein. In other embodiments, the peptides comprising a region of at least 8 amino acids including position 61 of the RAS peptide comprise positions 53 to 69 of the RAS protein. In alternative embodiments, the amino acid corresponding to position 61 of the RAS protein is at the C-terminus of the peptide. In further embodiments, the amino acid corresponding to position 61 of the RAS protein is at the N-terminus of the peptide. In general, the region having at least 8 amino acids including position 61 of the RAS protein may consist of any 8 positions of the RAS protein including position 13. For example, the region having at least 8 amino acids including position 61 may consist of the amino acids from position 54 to position 61, position 55 to position 62, position 56 to position 63, position 57 to position 64, position 58 to position 65, position 59 to position 66, position 60 to position 67 or position 61 to position 68 of the RAS protein.

The peptide mixtures of the present invention may contain the peptides in equal or in different proportions. In some embodiments, the first and second peptides are present in the mixture in equal proportions by absolute number of peptides, i.e. each peptide comprises 50% of the total number of peptides in the peptide component of the peptide mixture. In other embodiments, there is a greater proportion of the first peptide in the peptide mixture than the second peptide, by absolute number of peptides. For example, the first peptide may comprise at least 55%, at least 60%, at least 70%, at least 80% or at least 90% of the total number of peptides in the peptide component of the peptide mixture. In alternative embodiments, there is a greater proportion of the second peptide in the peptide mixture than the first peptide. For example, the second peptide may comprise at least 55%, at least 60%, at least 70%, at least 80% or at least 90% of the peptide component of the peptide mixture. In embodiments comprising at least one further peptide, the peptides are present in the peptide component of the peptide mixture in equal proportions. In other embodiments, the first, second and the at least one further peptide are present in different proportions from each other. For example, each of the first, second and at least one further peptide may independently comprise at least 1%, at least 5%, at least 10%, at least 20% at least 30%, at least 40%, at least 50%, at least 60%, at least 60%, at least 70%, at least 80% or at least 90% of the peptide component of the peptide mixture.

In a further embodiment, the peptides described above are for use as a vaccine or medicament. The peptide corresponds to a fragment of the RAS protein and comprises a region of at least 8 amino acids of the RAS protein including a mutated position, and wherein the mutated position is position 117 or 146 of the RAS protein. The region of at least 8 amino acids has at least 6 amino acid residues, other than at the mutated position, which are identical to the corresponding region of the RAS protein. The peptide for use as a vaccine or medicament has a point mutation at the mutated position, wherein the mutated position is position 117 or 146 of the RAS protein. In some embodiments, the point mutation of the peptide is one of a K117N, a A146T or a A146V mutation. As discussed above, it has now been found that peptides of the RAS protein having a point mutation at position 117 or 146, and particularly K117N, A146T and A146V peptides, are immunogenic and induce T-cell proliferation. In particular, FIG. 11 shows that a peptide having a mutation at position 146 of the RAS protein (more specifically, a A146T peptide) stimulates induction of T-cells.

Moreover, FIG. 12 shows that an immunogenic response to a peptide mixture is obtained even when the peptide mixture contains peptides originating from different regions of the wild-type RAS protein. Thus, FIG. 12 shows that there is no competition between the peptides of the mixture for binding to the HLA molecule responsible for presentation to the relevant T-cells, such immunodominance is not an important issue for the peptide mixtures of the invention.

The peptides of the present invention are peptides which correspond to the RAS protein fragments displayed by MHC II molecules on the surface of cells. Thus, the peptides of the present invention are peptides which correspond to the protein fragments which result from the intracellular proteolytic degradation of RAS proteins, which can then be displayed on HLA molecules, and to which individuals generally have a reactive T-cell in their T-cell repertoire.

In another aspect of the present invention, there is provided a T-cell, and a T-cell preparation comprising T-cells, specific for a peptide of the invention, as described above, wherein the peptide comprises a mutated position at position 117 or 146 of the RAS protein, when presented on an MHC molecule. In a further aspect of the present invention, there is provided a T-cell mixture comprising T-cells specific for each of the peptides in one of the peptide mixtures of the present invention.

The T-cell, T-cell preparation and T-cell mixture may be produced by stimulating at least one reactive T-cell with a peptide of the RAS protein, or peptide mixture comprising at least a first and a second peptide of the RAS protein. For example, in one embodiment, the T-cell or T-cell preparation is specific for a peptide corresponding to a fragment of the RAS protein, wherein the peptide comprises a region of at least 8 amino acids including a mutated position which is position 117 or 146 of the RAS protein, wherein the T-cell is specific for a point mutation at the mutated position of the peptide. In another embodiment, for example, the T-cell preparation comprises a plurality of T-cells specific for a peptide corresponding to a fragment of the RAS protein, wherein the peptide comprises a region of at least 8 amino acids including a mutated position, wherein the mutated position is position 117 or 146 of the RAS protein, and wherein each T-cell in the T-cell preparation is specific for a point mutation at the mutated position of the peptide. In another embodiment, for example, the T-cell mixture comprises a plurality of T-cells wherein a first and a second T-cell are specific for a first and a second peptide, respectively, corresponding to a fragment of the RAS protein, wherein each peptide comprises a region of at least 8 amino acids including a mutated position, wherein the mutated position of the first peptide is position 117 or 146 of the RAS protein, and the mutated position of the second peptide is position 12, 13, 61, 117 or 146 of the RAS protein, and wherein each of the first and second T-cells is specific for a point mutation at the amino acid corresponding to said mutated position, and the point mutation for which the first T-cell is specific is different from the point mutation for which the second T-cell is specific.

In a further aspect of the present invention, there is provided a T-cell receptor, or antigen-binding fragment thereof, specific for a peptide of the invention or a peptide of a peptide mixture of the invention, when presented on an MHC molecule. Also provided are T-cell receptors, or antigen-binding fragments thereof, specific for a peptide of the RAS protein, wherein the peptide comprises a region corresponding to a fragment of the RAS protein, wherein said region comprises at least 8 amino acids including a mutated position, said region comprises at least 6 amino acids, other than the mutated position, which are identical to the corresponding region of the RAS protein, wherein the mutated position has a point mutation, and the mutated position is position 12, 13 or 61 of the RAS protein. The antigen-binding fragment of a T-cell receptor can comprise a complete variable region of the T-cell receptor or the complementarity determining regions thereof in a suitable framework region.

In another aspect of the invention, there is provided a T-cell receptor mixture comprising at least a first and a second T-cell receptor, or antigen-binding fragment thereof, as described above. Also provided is a T-cell receptor mixture comprising at least a first and a second T-cell receptor, or antigen-binding fragment thereof, specific for a peptide of the RAS protein, wherein the peptide comprises a region which corresponds to a fragment of the RAS protein, wherein the region comprises at least 8 amino acids which includes a mutated position, the region comprises at least 6 amino acids, other than the mutated position, which are identical to the corresponding region of the RAS protein, wherein the mutated position has a point mutation, and the mutated position of each peptide is independently position 117, 146, 12, 13 or 61 of the RAS protein, and wherein each of the first and second T-cell receptor, or antigen-binding fragment thereof, is specific for a peptide having a different point mutation. In some embodiments, the T-cell receptor mixture comprises T-cell receptors, or antigen-binding fragments thereof, specific for each of the peptides in any of the peptide mixtures described above.

In another aspect of the present invention, there is provided mRNA encoding a T-cell receptor specific for the peptide of the invention or a peptide of a peptide mixture of the invention, when presented on an MHC molecule. In some embodiments, the mRNA is used to transfect host cells in order to display the encoded T-cell receptor on the host cell.

In another aspect of the present invention, there is provided a nucleic acid comprising a sequence which encodes the peptide of the present invention or a peptide of a peptide mixture of the present invention. There is also provided a mixture of nucleic acids, wherein each nucleic acid of the mixture comprises a sequence which encodes a different peptide of a peptide mixture of the present invention, such that the mixture of nucleic acids encodes a peptide mixture of the invention.

In some embodiments, the nucleic acids and mixtures thereof are used to synthesise the peptides or peptide mixtures of the present invention. For example, a peptide of the present invention may be synthesised by administering a nucleic acid to a subject, whereupon the nucleic acid is expressed by the subject, thereby giving rise to a peptide of the present invention in situ. The peptide produced then elicits an immune response in the subject. In another example, the nucleic acid may be used to synthesise a peptide of the present invention by transforming or transfecting a host cell with the nucleic acid of the present invention, such that the host cell expresses the nucleic acid to produce the peptide of the present invention which is then recovered and purified. In some embodiments, the peptides of the present invention are produced by chemical synthesis, using methods well known in the art.

In another aspect of the present invention, there is provided a vector comprising a nucleic acid comprising a sequence which encodes a peptide or a T-cell receptor of the present invention. In a further aspect, there is provided a host cell comprising a vector as described above. The host cell is transfected or transformed with the vector, such that the host cell expresses the nucleic acid encoded by the vector.

Peptides, peptide mixtures, T-cells, T-cell preparations, T-cell mixtures, T-cell receptors and antigen-binding fragments thereof, nucleic acids, vectors and host cells are for use in the treatment and/or prophylaxis of cancer, and in particular cancers associated with mutations in RAS oncogene. Cancers may include adrenal gland, autonomic ganglia, biliary tract, bone, breast, central nervous system, cervical, colorectal, endometrial, haematopoietic, lymphoid, kidney, large intestine, liver, lung, oesophagus, ovarian, pancreatic, prostate, salivary gland, skin, small intestine, stomach, testicular, thymus, thyroid, upper aerodigestive tract and urinary tract cancer, and malignant melanoma and the peptide mixtures, peptides, T-cell mixtures, T-cell preparations, nucleic acids, vectors and host cells of the present invention may be used for the prophylaxis and/or treatment of more than one of these types of cancer. In some embodiments, a peptide of the present invention, wherein the peptide has a A146T, a A146V or a K117N mutation, a peptide mixture of the present invention wherein the first peptide has a A146T, a A146V or a K117N mutation, a T-cell of the present invention wherein the T-cell is specific for a peptide having a A146T, a A146V or a K117N mutation, a T-cell preparation of the present invention wherein the T-cell is specific for a peptide having a A146T, a A146V or a K117N mutation, a T-cell mixture of the present invention wherein the first T-cell is specific for a peptide having a A146T, a A146V or a K117N mutation, a T-cell receptor or an antigen-binding fragment thereof of the present invention specific for a peptide having a A146T, a A146V or a K117N mutation, a nucleic acid of the present invention comprising a sequence encoding a peptide, or a T-cell receptor or an antigen-binding fragment thereof specific for a peptide, having a A146T, a A146V or a K117N mutation, a vector of the present invention comprising a nucleic acid encoding a peptide, or T-cell receptor or antigen-binding fragment thereof, specific for a peptide, having a A146T, a A146V or a K117N mutation, or a host cell of the present invention comprising a vector comprising a nucleic acid encoding a peptide, or T-cell receptor or antigen-binding fragment thereof, specific for a peptide, having a A146T, a A146V or a K117N mutation is for use in the prophylaxis and/or treatment of cancer. The T-cell receptors or antigen-binding fragments thereof are useful to engineer transgenic T-cells, for example, chimeric antigen receptor T-cells (CARTs). Such CARTs may be for use in personalised cancer therapy, for example, by intravenous infusion. In such embodiments, it is preferred that the cancer is one or more of colorectal, lung and pancreatic cancer. In some embodiments, the cancer is colorectal cancer. In particular, it has been found that, when selecting from the peptides and peptide mixtures of the present invention, 99% of cancers associated with mutations in the RAS protein can be treated.

More specifically, it has been found that a peptide mixture comprising a peptide having a A146T mutation, a peptide having a G13R mutation, a peptide having a G13V mutation, a peptide having a Q61R mutation, a peptide having Q61K mutation, a peptide having a Q61H mutation, and a peptide having a Q61L mutation can be used to treat 10% of all colorectal cancers whether or not they are associated with a mutation in RAS. Furthermore, it has been found that at least 50% of all colorectal cancers can be treated with either the above-mentioned peptide mixture or with a peptide mixture comprising a peptide having a G13C mutation, a peptide having a G13D mutation, a peptide having a G12A mutation, a peptide having a G12C mutation, a peptide having a G12D mutation, a peptide having a G12R mutation, a peptide having a G12S mutation, and a peptide having a G12V mutation (e.g. TG02). Thus, the peptides and peptide mixtures of the present invention provide vaccines and/or treatments for an increased number of colorectal cancer patients.

Pharmaceutical compositions comprising the peptides, peptide mixtures, T-cells, T-cell mixtures, T-cell preparations, or nucleic acids described above are also provided. Such pharmaceutical compositions may also comprise at least one pharmaceutically acceptable carrier, diluent and/or excipient. In some embodiments, the pharmaceutical composition further comprises one or more additional active ingredients and/or adjuvants. In certain embodiments the pharmaceutical composition may further comprise one or more ingredients therapeutically effective for the same disease indication. In one embodiment, the pharmaceutical composition of the present invention may further comprise one or more further chemotherapeutic agents, one or more antibodies, one or more small molecules and/or one or more immune stimulants (for example, cytokines). In some embodiments, the peptide, peptide mixture, T-cell, T-cell preparation, T-cell mixture, nucleic acid or the pharmaceutical composition may be used in combination with other forms of immunotherapy.

It has been found that certain types of cancer are associated with certain mutations of the RAS protein, and it has more recently been found that A146T, A146V and K117N mutations are associated with cancer, and particularly with colorectal cancer as shown in FIG. 1. Thus, it is possible to tailor the peptides, peptide mixtures, T-cells, T-cell preparations, T-cell mixtures, T-cell receptors or antigen-binding fragments thereof, nucleic acids, vectors and host cells to target certain types of cancer.

The peptide, peptide mixture, or pharmaceutical composition of the invention may be administered to a subject by any suitable delivery technique known to those skilled in the art. For example, among other techniques, the peptide, peptide mixture or pharmaceutical composition may be administered to a subject by injection, in the form of a solution, in the form of liposomes or in dry form (for example, in the form of coated particles, etc). In some embodiments, the peptide or peptide mixture is administered with an immune stimulator, such as GM-CSF. In embodiments using GM-CSF, this may be any GM-CSF. In some embodiments, the peptide, peptide mixture or pharmaceutical composition may be administered in an amount, for example, of between 1 μg and 1 g of each peptide once every three days, once a week, once a month, once every three months, once every four months or once every six months.

The T-cells, T-cell mixtures and T-cell preparations of the present invention may be administered by intra-venous injection and/or infusion, and may be administered in an amount, for example, of between 10⁶ and 10¹² of each T-cell specific for a peptide of the peptide mixture or peptide once every month, once every two months, once every three months, once every six months or once a year. Preferably, the dosage is administered once every month for between 2 and 5 months and is subsequently administered less frequently.

The nucleic acid and mixture of nucleic acids of the present invention may be administered by intra-muscular injection and/or subcutaneous injection.

Administration of a peptide or a peptide mixture of the present invention to a subject elicits an immune response to the peptide or peptide mixture, in particular a T-cell mediated immune response. The peptide, or each peptide of the peptide mixture, is processed by an antigen-presenting cell (APC) and is presented on an MHC molecule. T-cells are activated by binding of the T-cell receptor to a peptide presented on a MHC molecule by the APC, thereby resulting in an immune response against tumour cells having a mutation corresponding to that present in the administered peptide(s).

Administration of T-cells or T-cell mixtures of the present invention to subjects having a cancer associated with the corresponding RAS mutation elicits an immune response by the administered T-cells against the tumour cells. In particular, and as mentioned above, T-cells recognise peptides derived from intracellular proteins, such that the administered T-cells can recognise the mutated RAS proteins of the tumour cells, when displayed on the surface of the tumour cell by MHC molecules.

As mentioned above, the finding that different types of cancer are associated with different mutations of the RAS protein means that vaccines and treatments can be targeted to specific cancers. Thus, in another aspect of the invention, there is provided a peptide, peptide mixture, T-cell, T-cell mixture, T-cell preparation, a nucleic acid and/or a vector for use in a method comprising the diagnosis of cancer and the selection of an appropriate treatment. The method comprises the steps of a) identifying the RAS protein point mutations present in a sample taken from a patient, and b) selecting a peptide as described above, selecting a peptide mixture as described above comprising a peptide, selecting a T-cell as described above, selecting a T-cell mixture as described above comprising a T-cell specific for a peptide, selecting a T-cell preparation as described above comprising a T-cell specific for a peptide, selecting a nucleic acid as described above comprising a sequence encoding a peptide, and/or selecting a vector as described above encoding a nucleic acid comprising a sequence encoding a peptide, comprising a point mutation corresponding to at least one of the RAS protein point mutations identified in the sample. For example, if the sample taken from the subject is found to contain RAS proteins having a A146T mutation, then a peptide, a peptide mixture comprising a peptide, a T-cell, a T-cell mixture and/or preparation comprising a T-cell specific for a peptide, a nucleic acid comprising a sequence encoding a peptide, and/or a vector encoding a nucleic acid comprising a sequence encoding a peptide, comprising a A146T mutation is selected. In situations where the sample contains, for example, RAS proteins comprising a A146T mutation and a G13C mutation, a peptide mixture comprising a peptide comprising a A146T mutation and a peptide comprising a G13C mutation, a T-cell mixture comprising a T-cell specific for a peptide comprising a A146T and a T-cell specific for a peptide comprising a G13C mutation, a nucleic acid comprising a sequence encoding a peptide having a A146T mutation and a nucleic acid comprising a sequence encoding a peptide having a G13C mutation, and/or a vector encoding a nucleic acid comprising a sequence encoding a peptide having an A146T mutation and a vector encoding a nucleic acid comprising a sequence encoding a peptide having a G13C mutation is selected. The method may also comprise the step of administering a pharmaceutical composition comprising the selected peptide mixture, peptide, T-cell, T-cell mixture, T-cell preparation, nucleic acid and/or vector to the patient. Thus, in some embodiments, the method comprises the step of administering one of the peptide mixtures set out in Tables 6-23 and referred to herein as TG02 and TG03. In other embodiments, the method comprises the step of administering a T-cell mixture comprising T-cells specific for the peptide mixture of one of Tables 6-23, TG02 and TG03, nucleic acids encoding the peptide mixture of one of Tables 6-23, TG02 and TG03, and/or a vector or vectors encoding nucleic acids comprising a sequence or sequences encoding a peptide mixture of one of Tables 6-23, TG02 and TG03. In particularly preferred embodiments, the peptide mixture is one of TG02, TG03, the mixture of Table 14 and the mixture of Table 15.

In further aspects of the invention, there is provided a kit that includes a peptide, a peptide mixture, a T-cell, a T-cell mixture, a T-cell preparation, a nucleic acid, a nucleic acid mixture, a T-cell receptor, a T-cell receptor mixture, a vector, a host cell and/or mRNA as described herein. The peptide, peptide mixture, T-cell, T-cell mixture, T-cell preparation, nucleic acid, nucleic acid mixture, T-cell receptor, T-cell receptor mixture vector, host cell and/or mRNA as such may be present in the kit, or the peptide, peptide mixture, T-cell, T-cell mixture, T-cell preparation, nucleic acid, nucleic acid mixture, T-cell receptor, T-cell receptor mixture, vector, host cell and/or mRNA may be present in the kit as a pharmaceutical formulation. In some embodiments, the peptide, peptide mixture, T-cell, T-cell mixture, T-cell preparation, nucleic acid, nucleic acid mixture, T-cell receptor, T-cell receptor mixture, vector, host cell and/or mRNA may be packaged, for example in a vial, bottle, flask, which may be further packaged, for example, within a box, envelope or bag. In some embodiments, the kit comprises a peptide mixture, a T-cell mixture, nucleic acid mixture and/or T-cell receptor mixture wherein the peptides, the T-cells, the nucleic acids and/or the T-cell receptors are provided in separate containers, such that the peptides, T-cells, nucleic acids and/or the T-cell receptors are mixed by the user.

### Tables

TABLE 1
Position 146 mutated RAS
peptides of SEQ ID NOs: 1-4

| | |
|---|---|
| 138 146 154 | |
| GIPFIETSTKTRQRVED | (SEQ ID NO: 1) |
| GIPFIETSTKTRQGVED | (SEQ ID NO: 2) |
| GIPFIETSVKTRQRVED | (SEQ ID NO: 3) |
| GIPFIETSVKTRQGVED | (SEQ ID NO: 4) |

TABLE 2
Position 117 mutated RAS
peptides of SEQ ID NOs.: 5 and 6

| | |
|---|---|
| 109 117 125 | |
| VPMVLVGNNCDLPSRTV | (SEQ ID NO: 5) |
| VPMVLVGNNCDLPTRTV | (SEQ ID NO: 6) |

TABLE 3
Position 13 mutated RAS
peptides of SEQ ID NOs: 7-12

| | |
|---|---|
| 1 13 30 | |
| MTEYKLVVVGAGCVGKSALTIQLIQNHFVD | (SEQ ID NO: 7) |
| MTEYKLVVVGAGRVGKSALTIQLIQNHFVD | (SEQ ID NO: 8) |
| MTEYKLVVVGAGDVGKSALTIQLIQNHFVD | (SEQ ID NO: 9) |
| MTEYKLVVVGAGVVGKSALTIQLIQNHFVD | (SEQ ID NO: 10) |
| MTEYKLVVVGAGAVGKSALTIQLIQNHFVD | (SEQ ID NO: 11) |
| MTEYKLVVVGAGSVGKSALTIQLIQNHFVD | (SEQ ID NO: 12) |

TABLE 4
Position 12 mutated RAS
peptides of SEQ ID NOs: 13-18

| | |
|---|---|
| 1 12 30 | |
| MTEYKLVVVGAAGVGKSALTIQLIQNHFVD | (SEQ ID NO: 13) |
| MTEYKLVVVGACGVGKSALTIQLIQNHFVD | (SEQ ID NO: 14) |
| MTEYKLVVVGADGVGKSALTIQLIQNHFVD | (SEQ ID NO: 15) |
| MTEYKLVVVGARGVGKSALTIQLIQNHFVD | (SEQ ID NO: 16) |
| MTEYKLVVVGASGVGKSALTIQLIQNHFVD | (SEQ ID NO: 17) |
| MTEYKLVVVGAVGVGKSALTIQLIQNHFVD | (SEQ ID NO: 18) |

TABLE 5
Position 61 mutated RAS
peptides of SEQ ID NOs: 19-24

| | |
|---|---|
| 47 61 76 | |
| DGETCLLDILDTAGREEYSAMRDQYMRTGE | (SEQ ID NO: 19) |
| DGETCLLDILDTAGKEEYSAMRDQYMRTGE | (SEQ ID NO: 20) |
| DGETCLLDILDTAGHEEYSAMRDQYMRTGE | (SEQ ID NO: 21) |
| DGETCLLDILDTAGLEEYSAMRDQYMRTGE | (SEQ ID NO: 22) |
| DGETCLLDILDTAGEEEYSAMRDQYMRTGE | (SEQ ID NO: 23) |
| DGETCLLDILDTAGPEEYSAMRDQYMRTGE | (SEQ ID NO: 24) |

TABLE 6
Peptides contained in one embodiment
of the peptide mixture of the invention

| | |
|---|---|
| 138 146 154 | |
| GIPFIETSTKTRQRVED | (SEQ ID NO: 1) |
| 5 21 | |
| KLVVVGACGVGKSALTI | (SEQ ID NO: 28) |
| KLVVVGADGVGKSALTI | (SEQ ID NO: 29) |
| KLVVVGAVGVGKSALTI | (SEQ ID NO: 32) |
| KLVVVGAGDVGKSALTI | (SEQ ID NO: 26) |

TABLE 7
Peptides contained in one embodiment
of the peptide mixture of the invention

138 146 154

| | |
|---|---|
| GIPFIETSTKTRQGVED | (SEQ ID NO: 2) |
| 5 21 | |
| KLVVVGACGVGKSALTI | (SEQ ID NO: 28) |
| KLVVVGADGVGKSALTI | (SEQ ID NO: 29) |
| KLVVVGAVGVGKSALTI | (SEQ ID NO: 32) |
| KLVVVGAGDVGKSALTI | (SEQ ID NO: 26) |

TABLE 8
Peptides contained in one embodiment
of the peptide mixture of the invention

138 146 154

| | |
|---|---|
| GIPFIETSTKTRQRVED | (SEQ ID NO: 1) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 9
Peptides contained in one embodiment
of the peptide mixture of the invention

138 146 154

| | |
|---|---|
| GIPFIETSTKTRQGVED | (SEQ ID NO: 2) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 10
Peptides contained in one embodiment of the
peptide mixture of the invention
(TG02 + KRAS A146T)

| | |
|---|---|
| 138 146 154 | |
| GIPFIETSTKTRQRVED | (SEQ ID NO: 1) |
| 5 21 | |
| KLVVVGAGCVGKSALTI | (SEQ ID NO: 25) |
| KLVVVGAGDVGKSALTI | (SEQ ID NO: 26) |
| KLVVVGAAGVGKSALTI | (SEQ ID NO: 27) |
| KLVVVGACGVGKSALTI | (SEQ ID NO: 28) |
| KLVVVGADGVGKSALTI | (SEQ ID NO: 29) |
| KLVVVGARGVGKSALTI | (SEQ ID NO: 30) |
| KLVVVGASGVGKSALTI | (SEQ ID NO: 31) |
| KLVVVGAVGVGKSALTI | (SEQ ID NO: 32) |

TABLE 11
Peptides contained in one embodiment of
the peptide mixture of the invention
(TG02 + NRAS A146T)

| | |
|---|---|
| 138 146 154 | |
| GIPFIETSTKTRQGVED | (SEQ ID NO: 2) |
| 5 21 | |
| KLVVVGAGCVGKSALTI | (SEQ ID NO: 25) |
| KLVVVGAGDVGKSALTI | (SEQ ID NO: 26) |
| KLVVVGAAGVGKSALTI | (SEQ ID NO: 27) |
| KLVVVGACGVGKSALTI | (SEQ ID NO: 28) |
| KLVVVGADGVGKSALTI | (SEQ ID NO: 29) |
| KLVVVGARGVGKSALTI | (SEQ ID NO: 30) |
| KLVVVGASGVGKSALTI | (SEQ ID NO: 31) |
| KLVVVGAVGVGKSALTI | (SEQ ID NO: 32) |

TABLE 12
Peptides contained in one embodiment of
the peptide mixture of the invention
(TG02 + NRAS A146V)

| | |
|---|---|
| 138 146 154 | |
| GIPFIETSVKTRQGVED | (SEQ ID NO: 4) |
| 5 21 | |
| KLVVVGAGCVGKSALTI | (SEQ ID NO: 25) |
| KLVVVGAGDVGKSALTI | (SEQ ID NO: 26) |
| KLVVVGAAGVGKSALTI | (SEQ ID NO: 27) |
| KLVVVGACGVGKSALTI | (SEQ ID NO: 28) |
| KLVVVGADGVGKSALTI | (SEQ ID NO: 29) |
| KLVVVGARGVGKSALTI | (SEQ ID NO: 30) |
| KLVVVGASGVGKSALTI | (SEQ ID NO: 31) |
| KLVVVGAVGVGKSALTI | (SEQ ID NO: 32) |

TABLE 13
Peptides contained in one embodiment of the
peptide mixture of the invention
(TG02 + KRAS A146V)

| | |
|---|---|
| 138 146 154 | |
| GIPFIETSVKTRQRVED | (SEQ ID NO: 3) |
| 5 21 | |
| KLVVVGAGCVGKSALTI | (SEQ ID NO: 25) |
| KLVVVGAGDVGKSALTI | (SEQ ID NO: 26) |
| KLVVVGAAGVGKSALTI | (SEQ ID NO: 27) |
| KLVVVGACGVGKSALTI | (SEQ ID NO: 28) |
| KLVVVGADGVGKSALTI | (SEQ ID NO: 29) |
| KLVVVGARGVGKSALTI | (SEQ ID NO: 30) |
| KLVVVGASGVGKSALTI | (SEQ ID NO: 31) |
| KLVVVGAVGVGKSALTI | (SEQ ID NO: 32) |

TABLE 14
Peptides contained in one embodiment of the
peptide mixture of the invention
(TG03 + KRAS A146T)

| | |
|---|---|
| 138 146 154 | |
| GIPFIETSTKTRQRVED | (SEQ ID NO: 1) |
| 5 21 | |
| KLVVVGAGRVGKSALTI | (SEQ ID NO: 33) |
| KLVVVGAGVVGKSALTI | (SEQ ID NO: 34) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 15
Peptides contained in one embodiment of the
peptide mixture of the invention
(TG03 + NRAS A146T)

| | |
|---|---|
| 138 146 154 | |
| GIPFIETSTKTRQGVED | (SEQ ID NO: 2) |
| 5 21 | |
| KLVVVGAGRVGKSALTI | (SEQ ID NO: 33) |
| KLVVVGAGVVGKSALTI | (SEQ ID NO: 34) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 16
Peptides contained in one embodiment of the
peptide mixture of the invention
(TG03 + KRAS A146V)

| | |
|---|---|
| 138 146 154 | |
| GIPFIETSVKTRQRVED | (SEQ ID NO: 3) |
| 5 21 | |
| KLVVVGAGRVGKSALTI | (SEQ ID NO: 33) |
| KLVVVGAGVVGKSALTI | (SEQ ID NO: 34) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 17
Peptides contained in one embodiment of the
peptide mixture of the invention
(TG03 + NRAS A146V)

| | |
|---|---|
| 138 146 154 | |
| GIPFIETSVKTRQGVED | (SEQ ID NO: 4) |
| 5 21 | |
| KLVVVGAGRVGKSALTI | (SEQ ID NO: 33) |
| KLVVVGAGVVGKSALTI | (SEQ ID NO: 34) |
| 53 59 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 18
Peptides contained in one embodiment of the
peptide mixture of the invention
(TG02 + KRAS K117N)

| | |
|---|---|
| 109 117 125 | |
| VPMVLVGNNCDLPSRTV | (SEQ ID NO: 5) |
| 5 21 | |
| KLVVVGAGCVGKSALTI | (SEQ ID NO: 25) |
| KLVVVGAGDVGKSALTI | (SEQ ID NO: 26) |
| KLVVVGAAGVGKSALTI | (SEQ ID NO: 27) |
| KLVVVGACGVGKSALTI | (SEQ ID NO: 28) |
| KLVVVGADGVGKSALTI | (SEQ ID NO: 29) |
| KLVVVGARGVGKSALTI | (SEQ ID NO: 30) |
| KLVVVGASGVGKSALTI | (SEQ ID NO: 31) |
| KLVVVGAVGVGKSALTI | (SEQ ID NO: 32) |

TABLE 19
Peptides contained in one embodiment of the
peptide mixture of the invention
(TG02 + NRAS K117N)

| | |
|---|---|
| 109 117 125 | |
| VPMVLVGNNCDLPTRTV | (SEQ ID NO: 6) |
| 5 21 | |
| KLVVVGAGCVGKSALTI | (SEQ ID NO: 25) |
| KLVVVGAGDVGKSALTI | (SEQ ID NO: 26) |
| KLVVVGAAGVGKSALTI | (SEQ ID NO: 27) |
| KLVVVGACGVGKSALTI | (SEQ ID NO: 28) |
| KLVVVGADGVGKSALTI | (SEQ ID NO: 29) |
| KLVVVGARGVGKSALTI | (SEQ ID NO: 30) |
| KLVVVGASGVGKSALTI | (SEQ ID NO: 31) |
| KLVVVGAVGVGKSALTI | (SEQ ID NO: 32) |

TABLE 20
Peptides contained in one embodiment of the
peptide mixture of the invention
(TG03 + KRAS K117N)

| | |
|---|---|
| 109 117 125 | |
| VPMVLVGNNCDLPSRTV | (SEQ ID NO: 5) |
| 5 21 | |
| KLVVVGAGRVGKSALTI | (SEQ ID NO: 33) |
| KLVVVGAGVVGKSALTI | (SEQ ID NO: 34) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 21
Peptides contained in one embodiment of the
peptide mixture of the invention
(TG03 + NRAS K117N)

| | |
|---|---|
| 109 117 125 | |
| VPMVLVGNNCDLPTRTV | (SEQ ID NO: 6) |
| 5 21 | |
| KLVVVGAGRVGKSALTI | (SEQ ID NO: 33) |
| KLVVVGAGVVGKSALTI | (SEQ ID NO: 34) |
| 53 69 | |
| LDILDTAGREEYSAMRD | (SEQ ID NO: 35) |
| LDILDTAGKEEYSAMRD | (SEQ ID NO: 36) |
| LDILDTAGHEEYSAMRD | (SEQ ID NO: 37) |
| LDILDTAGLEEYSAMRD | (SEQ ID NO: 38) |

TABLE 22
Peptides contained in one embodiment of the
peptide mixture of the invention (TGX3)

| | |
|---|---|
| 138 146 154 | |
| GIPFIETSTKTRQRVED | (SEQ ID NO: 1) |
| 5 21 | |
| KLVVVGAGCVGKSALTI | (SEQ ID NO: 25) |
| KLVVVGAGDVGKSALTI | (SEQ ID NO: 26) |
| 53 69 | |
| LDILDTAGREEYSAMRD | (SEQ ID NO: 35) |

TABLE 23
Peptides contained in one embodiment of the
peptide mixture of the invention
(alternative form of TGX3)

| | |
|---|---|
| 138 146 154 | |
| GIPFIETSTKTRQGVED | (SEQ ID NO: 2) |
| 5 21 | |
| KLVVVGAGCVGKSALTI | (SEQ ID NO: 25) |
| KLVVVGAGDVGKSALTI | (SEQ ID NO: 26) |
| 53 69 | |
| LDILDTAGREEYSAMRD | (SEQ ID NO: 35) |

### EXAMPLES

### Example 1

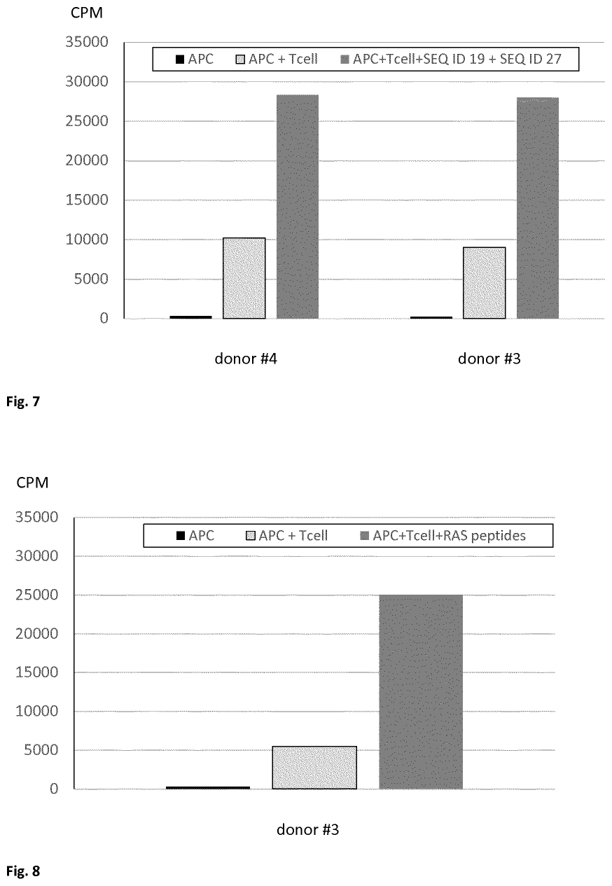

In this example, Buffy coats were collected from 4 normal human donors (Buffy 1, Buffy 2, Buffy 3, and Buffy 4) and were cultured in vitro. The in vitro PBMCs were stimulated with a single RAS peptide or a mixture of RAS peptides, and T-cell proliferation assays performed. The results are shown in FIGS. 7-9.

### Method

Equipment/Reagents
Hettich Rotina 420 (radius 210) or equivalent
KOJAIR Silverline Blue Series laminar flow hood or equivalent
CO₂ incubator, Forma Scientific Model 3111 or equivalent
Water bath 37° C.
KOVA Glasstic slide (Cat no. 87144E, Hycor Biomedical Inc, Garden Grove, USA)
TopCount, Microplate scintillation counter (Packard Instrument Company, Meriden, USA)
Cell Harvester Filtermate 196 Harvester, (Packard Instrument Company, Meriden, USA)
Unifilter GF/C (Cat.no. 6-005174, Nerliens Meszansky, Oslo, Norway) or equivalent
Microscint-0 scintillation liquid (Cat. No. 6013611, Nerliens Meszansky, Oslo, Norway) or equivalent
Topseal-A (Cat. No. 6005185, Nerliens Meszansky, Oslo, Norway) or equivalent
³H-Thymidine (Cat no. ART178-D, Nerliens Meszansky, Oslo, Norway) or equivalent
CellGro DC medium (Cat. no. 0020801-0500, CellGenix GmbH, Freiburg, Germany) or equivalent
RPMI-1640 (Cat no: E15-840) PAA Labs, Linz, Austria) or equivalent
Dimethylsulfoxide (DMSO) (Cat no: D5879-500ML, Sigma-Aldrich Norway AS, Oslo, Norway) or equivalent
Mucomyst (Cat.no. 019249, Meda AS, Asker, Norway) or equivalent
Recombinant human interleukin-2 (IL-2, Proleukin®), (Chiron Therapeutics, Emeryville, USA) or equivalent
4-(2-hydroxyethyl)-1-piperazineethanesulfonic acid (HEPES) buffer 1M (Cat. No. S11-001, Fisher Scientific AS, Oslo, Norway) or equivalent
IL-7 (Cat no. 207-IL-025, R & D Systems Europe Ltd, Abingdon, UK) or equivalent
Gentamicin 40 mg/ml (Cat. No. Sanofi Aventis Norge AS, Lysaker, Norway) or equivalent
Human Serum Albumin 20%, (Cat. No. SW2G0013, Baxter AS, Oslo, Norway) or equivalent
24-well tissue culture plates (Cat. No. 734-1605, VWR International AS, Oslo, Norway) or equivalent
Microplate 96-well, round bottomed (Cat. No. 734-1797, VWR International AS, Oslo, Norway) or equivalent
Staphylococcal enterotoxin type C (SEC-3) (Cat. No. CT333, Toxin Technology Inc, Sarasota, USA).

Complete CellGro DC Medium Used for Culture:

The following was added to 500 ml of CellGro DC medium for the final concentrations: Gentamicin 50 μg/ml (add 630 μL of 40 mg/ml stock to 500 mL medium) Mucomyst 1.6 mg/ml (add 4 mL of 200 mg/ml stock to 500 mL medium) HEPES buffer 0.01M (add 5 ml of 1M stock to 500 mL of medium)

Procedure

a. Thawing of Frozen PBMC

The procedure must be performed at room temperature until point 5. All handling of cells in the open is done in a vertical laminar flow hood.
1. Rapidly transfer the vials, each vial with frozen PBMC from a buffy coat (Buffy 1, Buffy 2, Buffy 3, and Buffy 4), to a water bath at 37° C.
2. Shake the vials manually at regular intervals (approx. 2-3 min.) and remove them from the water bath while some ice is still present.
3. When all the ice is melted, transfer 1 ml of CellGro DC medium drop-wise to the cell suspension.
4. Transfer the cell suspension to a 50 ml tube containing 20 ml of CellGro DC medium.
5. Centrifuge cells at 500G for 5 min at room temperature.
6. Resuspend the cells in 5 ml CellGro DC medium.
7. Count the number of viable cells using a Burker chamber or KOVA Galsstic slides and adjust the cell concentration to 2×10⁶ cells/ml in complete CellGro DC medium (see recipe). Total cell numbers: Buffy 1-45×10⁶, Buffy 2-27.5×10⁶, Buffy 3-40.5×10⁶, and Buffy 4-40.5×10⁶ cells.

b. Bulk cultures for increasing number of RAS peptide reactive T-cells
1. Transfer 1 ml of thawed PBMCs (2×10⁶ cells/ml in DC medium) to each well in a 24-well plate.

TABLE 24
Re-stimulation: Number of wells stimulated with the peptide mixes.

| | Total number of cells | Peptide mix: | Peptide mix: |
|---|---|---|---|
| | (mill) | 13C + 13R | 13R + TG02-mix |
| Buffy 1 | 45 | 11 | 11 |
| Buffy 2 | 27.5 | 6 | 6 |
| Buffy 3 | 40.5 | 10 | 10 |
| Buffy 4 | 40.5 | 10 | 10 |

2. Add 20 μl of each of the 13C and 13R peptides, or 20 μl 13R and 60 μl TG02-mix to the wells for a final concentration of 10 μM of each peptide.
3. Culture the cells in a humidified incubator at 37° C./5% CO₂ for 3 days
4. Day 3: Add a final concentration of 20 iU/ml of recombinant human interleukin-2 (rIL-2) (i.e. 50 μl from stock solution of 1000 iU/ml) and final concentration of
5 ng/ml recombinant human IL-7 (i.e. 10 μl from stock solution of 500 μg/ml) to the cell cultures and continue incubation at 37° C./5% CO₂. This step is optional if the cells are growing well.
5. Day 4-6: Cells are checked regularly under the microscope and split when required (500 μl was withdrawn from each well and replaced with 500 μl fresh CellGro DC medium, supplemented with 40 iU/ml IL-2 and IL-7).
6. Day 7-14: Cells are checked each day and wells with slow growing cells are mixed together.

c. i) 3-Days T-Cell Proliferation Assay
1. Harvest, wash and count T-cells in the bulk cultures from step b.;

TABLE 25
Total T-cell numbers

| | Peptide mix: 13C + 13R | Peptide mix: 13R + TG02-mix |
|---|---|---|
| Buffy 1 | 0.9 × 10⁶ | 0.72 × 10⁶ |
| Buffy 2 | 5.0 × 10⁶ | 4.05 × 10⁶ |
| Buffy 3 | 7.65 × 10⁶ | 7.2 × 10⁶ |
| Buffy 4 | 6.3 × 10⁶ | 2.7 × 10⁶ |

2. Transfer 5×10⁴ T-cells from bulk cultures per well in round-bottomed 96-well plates.
3. Thaw 1 vial of autologous PBMCs sample in CellGro DC medium. Irradiate PBMCs (30 Gy), count and add 5×10⁴ cells to each well and adjust to a total volume of 200 μl/well with DC medium.
4. Prepare the following samples in triplicates, according to plate layout:
   Negative controls:
   T-cells only
   T-cells from each time point+irradiated PBMC
   Positive control:
   T-cells from each time point+irradiated PBMC+1 μg/ml SEC-3.
   Test sample:
   T-cells from each time point+irradiated PBMC (10 μM of each peptide):
      I) For bulk cultures stimulated with 13C+13R: 13C+13R mix or single G13C peptides
      II) For bulk cultures stimulated with TG02+13R: TG02+13R, 13C+13R mix, or single G13C peptides
   Incubate the cells for 48 hours at 37° C./5% CO₂.
5. Add 20 μL of ³H-Thymidine (3.7×10⁴ Bq).
6. Incubate at 37° C./5% CO₂ for 17 hours.
7. Harvest the cells to Unifilters using the Filtermate 196 Harvester and dry the filters at 45° C. until completely dry (normally this is achieved after 1.5 but the number of hours left at 45° C. after this is not critical, hence plates can be counted 60 hours later).
8. Cover the bottom of the Unifilters with adhesive covers (delivered with the Unifilters) and add 25 μl micro scintillation liquid to each well. Cover the plate with TopSeal and place the filters in a TopCount Packard microplate scintillation beta counter. Enter assay wizard program. Select protocol/program ³H Thymidine in triplicates. Enter report definition and ASCII file output. Under directory, select data folder (each user should have a separate folder). Choose name for experiment file to save. Stacker on or off (use stacker if more than one plate). Start the assay program.

ii) Second Stimulation of Bulk Cultures

The remaining cells (1-2×10⁶ T-cells/well) were re-stimulated once more with autologous PBMCs (1 mill/well) and peptide mixes (as described in step b.).

TABLE 26
Stimulation - Numbers of wells stimulated with the peptide mixes

| | Peptide mix: 13C + 13R | Peptide mix: 13R + TG02-mix |
|---|---|---|
| Buffy 1 | 1 | 1 |
| Buffy 2 | 2 | 2 |
| Buffy 3 | 3 | 3 |
| Buffy 4 | 3 | 1 |

1. Culture the cells in a humidified incubator at 37° C./5% CO₂ for 3 days (as described in step b.).
2. Day 17: Add a final concentration of 40 iU/ml of recombinant human interleukin-2 and final concentration of 5 ng/ml recombinant human IL-7 to the cell cultures and continue incubation at 37° C./5% CO₂. Cells are checked regularly under the microscope and split when required.
3. Day 19-21: 500 μl was withdrawn from each well and replaced with 500 μl fresh CellGro DC medium, supplemented with 40 iU/ml IL-2 and IL-7.
4. Day 22-27: Cells were checked regularly each day, and wells with slow growing T-cells were mixed together (as in step b.).

d. i) 3-Days T-Cell Proliferation Assay
1. Harvest, wash and count T-cells in the bulk cultures.

TABLE 27
Total cell number

| | Peptide mix: 13C + 13R | Peptide mix: 13R + TG02-mix |
|---|---|---|
| Buffy 1 | 0.09 × 10⁶ | 0.18 × 10⁶ |
| Buffy 2 | 4.5 × 10⁶ | 5.4 × 10⁶ |
| Buffy 3 | 2.7 × 10⁶ | 3.15 × 10⁶ |
| Buffy 4 | 1.35 × 10⁶ | 1.76 × 10⁶ |

2. Transfer 5×10⁴ T-cells from bulk cultures per well in round-bottomed 96-well plates.
3. Thaw 1 vial of autologous PBMCs sample in CellGro DC medium. Irradiate PBMCs (30 Gy), count and add 5×10⁴ cells to each well and adjusted to a total volume of 200 μl/well with DC medium (as described in step c.i)).

ii) Day 27-42: Third Stimulation of Bulk Cultures

The remaining cells (1-2 mill T-cells/well) were re-stimulated once more with autologous PBMs (1 mill/well) and peptide mix (as described in step b.)

TABLE 28
Stimulation - Numbers of wells stimulated

| | Peptide mix: 13C + 13R | Peptide mix: 13R + TG02-mix |
|---|---|---|
| Buffy 1 | 0 | 0 |
| Buffy 2 | 2 | 2 |
| Buffy 3 | 1 | 1 |
| Buffy 4 | 1 | 1 |

e) 3-Days T-Cell Proliferation Assay
1. Harvest, wash and count T-cells in the bulk cultures.

TABLE 29
Total cell number

| | Peptide mix: 13C + 13R | Peptide mix: 13R + TG02-mix |
|---|---|---|
| Buffy 1 | 0 | 0 |
| Buffy 2 | 0.9 × 10⁶ | 0.63 × 10⁶ |
| Buffy 3 | 0.27 × 10⁶ | 0.675 × 10⁶ |
| Buffy 4 | 0.315 × 10⁶ | 0.18 × 10⁶ |

2. Transfer 5×10⁴ T-cells from bulk cultures per well into round-bottomed 96-well plates.
3. Thaw 1 vial of autologous PBMCs sample in CellGro DC medium. Irradiate PBMCs (30 Gy), count and 5×10⁴ cells were added to each well and adjusted to a total volume of 200 μl/well with DC medium (as described in step c.i)).

Results

The results of the T-cell proliferation assays with the peptide mixtures, following three rounds of stimulation of the bulk cultures, are shown in Tables 30-32, and FIGS. 7-9, respectively. Tables 30-32 show the counts per minute (CPM) for each replicate, and the mean CPM for each donor.

TABLE 30
Counts per minute (CPM) after stimulation with a peptide mixture
consisting of 13C and 13R peptides. During the three rounds of in vitro
stimulation of the bulk cultures, Donor #3 was stimulated with the
TG02 + 13R peptide mixture, while Donor #4 was stimulated
with the 13C + 13R peptide mixture.

| | APC | APC + T-cell | APC + T-cell + 13 C + 13 R |
|---|---|---|---|
| Donor #3 | 337 | 8337 | 28,422 |
| | 235 | 12,061 | 28,264 |
| | 375 | n/a | n/a |
| Mean Donor #3 | 316 | 10,199 | 28,343 |
| Donor #4 | 137 | 9696 | 32,476 |
| | 421 | 8412 | 23,499 |
| | 249 | n/a | n/a |
| Mean Donor #4 | 269 | 9054 | 27,987 |

TABLE 31
Counts per minute (CPM) after stimulation with a peptide mixture
consisting of 13C, 13D, 12A, 12C, 12D, 12R, 12S, 12V and 13R (i.e.
TG02 + 13R) peptides. During the three rounds of in vitro stimulation
of the bulk cultures, Donor #3 was stimulated with the
TG02 + 13R peptide mixture.

| | APC | APC + T-cell | APC + T-cell + 13R + TG02 |
|---|---|---|---|
| Donor #3 | 337 | 4954 | 28,796 |
| | 235 | 6073 | 21,201 |
| | 375 | 5513 | n/a |
| Mean Donor #3 | 316 | 5513 | 24,998 |

TABLE 32
Counts per minute (CPM) after stimulation with a 13C peptide. During
the three rounds of in vitro stimulation of the bulk cultures, Donor #3 was
stimulated with the TG02 + 13R peptide mixture.

| | APC | APC + T-cell | APC + T-cell + 13 C |
|---|---|---|---|
| Donor #3 | 337 | 4954 | 27813 |
| | 235 | 6073 | 32522 |
| | 375 | 5513 | 28869 |
| Mean Donor #3 | 316 | 5513 | 29735 |

The results of the positive control were confirmed but are not included in FIGS. 7-9 for scaling reasons. As can be seen, both of the peptide mixtures and the single peptide induced T-cell proliferation, indicating that the peptide and peptide mixtures were able to induce an immune response in humans.

### Example 2

In this Example, mice were repeatedly vaccinated subcutaneously with TG02, in order to analyse the immune response. Following the vaccination, splenocytes were harvested, and the proliferative response of the splenocytes was measured. The results are shown in FIG. 10.

### Method

Characterisation of the Test Item

Name: TG02

Product: TG02 consists of equal amounts (weight) of 8 different peptides (12A, 12C, 12D, 12R, 12S, 12V, 13C, 13D)

Batch No.: 12A: lot no 1034804; 12C: lot no 1034803;
12D: lot no 1034801; 12R: lot no 1034802;
12S: lot no 1034805; 12V: lot no 1034800;
13C: lot no 1050468; 13D: lot no 1034806

Therapeutic Indication: cancer

Physical State: powder

Colour: white

Purity: 80 mg net peptide per vial (10 mg net of each peptide)

Storage Conditions: −15° C.-−20° C. and protected from light

Expiry Date: 31 Dec. 2014

Safety Precautions: Routine hygienic procedures were sufficient to assure personnel

health and safety.

Characterisation of the Vehicle 1

Name: ViscoGel

Batch No.: VG14056

Therapeutic Indication: cancer

Physical State: gel particles

Colour:colourless

Water Content: 99%

Storage Conditions: 2-8° C.

Expiry Date: 1 Jun. 2015

Safety Precautions: The routine hygienic procedures will be sufficient to assure

personnel health and safety.

Characterisation of the Vehicle 2

The vehicle 2 to be used in this study will be aqua ad injectionem. The specifications provided by the supplier are listed as follows:

Name: aqua ad injectionem

Physical State: liquid

Storage Conditions: room temperature

Safety Precautions: Routine hygienic procedures were sufficient to assure personnel health and safety.

Preparation of the Test Item

5 ml aqua ad injectionem was added to one vial TG02 (80 mg) and was gently swirled (avoid foaming) to obtain a homogenous stock solution of 16 mg TG02 per mL.

For animals of group 1 (see Table 28), 1 mL TG02 stock solution was extracted with a syringe and mixed with 1 mL aqua ad injectionem to obtain a final concentration of 8 mg/mL.

For animals of the groups 2 and 3 (see Table 28), 1 ml TG02 stock solution was extracted with a syringe and mixed with 1 mL ViscoGel™ to obtain a final concentration of 8 mg/mL.

Test item formulations were considered to be stable for 6 h at 2-8° C.

Test System

Species/strain: healthy BALB/c mice (full barrier) BALB/cAnNCrl

Source: Charles River, 97633 Sulzfeld, Germany

Sex: female

Age at the start of the treatment period: approximately 6-8 weeks old

Number of animals: 30 (10 animals per group)

The animals were derived from a controlled full-barrier maintained breeding system (SPF). According to Art. 9.2, No. 7 of the German Act on Animal Welfare, the animals were bred for experimental purposes.

Housing and Feeding Conditions
Full barrier in an air-conditioned room.
Temperature: 22±3° C.
Relative humidity: 55±10%.
Artificial light, sequence being 12 hours light, 12 hours dark.
Air change: 10×/hour.
Free access to Altromin 1324 maintenance diet for rats and mice.
Free access to tap water, sulphur acidified to a pH of approximately 2.8 (drinking water, municipal residue control, microbiological controls at regular intervals).

The animals will be kept in groups of 5 animals per cage in IVC cages, type II L, polysulphone cages on Altromin saw fibre bedding.
Certificates of food, water and bedding are filed at BSL BIOSERVICE.
Adequate acclimatisation period (at least 5 days) under laboratory conditions.

Allocation and Identification of the Animals Animals showing pathological signs before administration were excluded from the study. Supplementary animals from the same delivery were provided in exchange. Each animal was marked for individual identification with an ear mark.

Experimental Procedure

The study was conducted with 3 groups, each compromising 10 female BALB/c mice. The start of the study was performed on two separate days on which 5 of the animals per group were treated. Therefore the groups were divided into part I and part II. The animals were treated subcutaneously at different time points (Table 30).

During the period of administration, the animals were observed precisely each day for signs of toxicity. 48 hours after the last administration the animals were euthanised, examined macroscopically and the spleen was prepared for further analysis.

Dosage

In all groups the test item was administered at repeated time points (Table 33) by subcutaneous injection between the nape of the neck and the shoulder. The application volume for all groups was 0.1 mL (0.80 mg TG02).

TABLE 33
Treatment and Animal Identification

| | | | | | Subjected to |
|---|---|---|---|---|---|
| | | | | | Necropsy |
| | | | | Time Points of | (hours after |
| | | Animal | Animal | Subcutaneous | last |
| | | No. | No. | Application | admin- |
| Group | Treatment | Part I | Part II | (Day) | istration) |
| 1 | TG02 | 1-5 | 6-10 | 1, 8, 15, 22 | 48 |
| 2 | TG02 + | 11-15 | 16-20 | 1, 8, 15, 22 | 48 |
| | ViscoGel ™ | | | | |
| 3 | TG02 + | 21-25 | 26-30 | 1, 22 | 48 |
| | ViscoGel ™ | | | | |

Clinical Observations

All animals were observed for clinical signs during the entire treatment period of 24 days.

General clinical observations were made at least once a day, preferably at the same time each day and considering the peak period of anticipated effects after dosing. The health condition of the animals was recorded.

On each of the animals, general clinical observations including changes in the skin and fur, eyes and mucous membranes were performed preferably at the same time each day and considering the peak period of anticipated effects after dosing. Also respiratory, circulatory, autonomic and central nervous systems and somatomotor activity and behaviour pattern were examined. Particular attention was directed to observations of signs of anaphylactoid reactions, paralysis, tremor, convulsions, salivation, diarrhoea, lethargy, sleep and coma. Moreover, attention was directed to the injection site.

Pathology—Gross Necropsy

48 hours after the last administration (study day 24) animals were sacrificed by cervical dislocation and were subjected to a detailed gross necropsy which includes careful examination of the external surface of the body, all orifices and the cranial, thoracic and abdominal cavities and their contents.

Cell Culture and Stimulation of Splenic Cells

The spleen of all animals was removed, transferred to cell culture medium (RPMI 1640 medium supplemented with 10% FCS, 100 U/mL Penicillin, 100 μg/mL Streptomycin, 2 mM L-Glutamine, and 50 μM beta-mercaptoethanol) and stored on ice. All steps were performed sterile and cells were kept on ice.

A single cell suspension from splenic cells was generated using a cell strainer. After centrifugation (350 g, 5 min, 5±3° C.), supernatant was removed and the cell pellet was resuspended in ACK buffer and incubated for 5 min at RT. 10 mL cell culture medium was added. The samples were left 5 min on bench top to let the cell debris sediment. The suspension above the cell debris was transferred into another tube and was centrifuged (350 g, 5 min, 5±3° C.). The supernatant was removed and the cell pellet was resuspended in 10 mL cell culture medium. After counting of cells, 0.2 Mio cells were seeded in a 96 well plate (180 μL per well; 1,1*10⁶ cells/mL). 9 replicates were plated on each plate for each spleen.

(1) 1 plate for harvesting of supernatant for cytokine measurement after 24 h (results not shown)

(2) 1 plate for harvesting of supernatant for cytokine measurement after 48 h (results not shown)

(3) 1 plate for the proliferation assay

The following stimulations were performed:
3 replicates: unstimulated (addition of 20 μL cell culture medium)
3 replicates: stimulation with 10 μM TG02 (8 peptides, 10 μM end concentration per peptide, addition in 20 μL cell culture medium)
3 replicates: stimulation with 1 μg/200 μL ConA (Concanavalin A) (addition of 1 μg ConA in 20 μL cell culture medium)

Incubation at 37° C. and 5% CO₂ for 24 h (1), 48 h (2) or 5 days (3).

The remaining cells were centrifuged (350 g, 5 min, 5±3° C.), transferred to a 1.5 mL tube, centrifuged again (350 g, 5 min, 5±3° C.), the supernatants were completely removed and the cell pellets were frozen at ≤−70° C.
(1) After 24 h, the corresponding plates were centrifuged (350 g, RT) and 150 μL of the supernatant were harvested and frozen at ≤−70° C.
(2) After 48 h, the corresponding plates were centrifuged (350 g, RT) and 150 μL of the supernatant were harvested and frozen at ≤−70° C.

Proliferation Assay
(3) After 5 days, 1 μCi/well ³H-Thymidine was added to the samples, which were then incubated for 18 h at 37° C. and 5% CO₂. The plates were then frozen at 5-20° C.

Plates were thawed at RT. After washing off the harvester, the samples were transferred to a filter plate using the harvester followed by 5 washing steps using water. Filter plates were dried at RT overnight. A foil was stuck to the bottom of the filter plates and 20 μL scintillation fluid was added to the wells. After incubation of 1 h at RT, the samples were measured using a TopCount NXT and the stimulation index (SI) was calculated. SI=CPM of stimulated samples/CPM of control samples.

Results

Table 34 and FIG. 10 show the results of the splenocyte proliferation assay. Table 34 shows the CPM for each replicate, and the mean CPM for each mouse. As can be seen, splenocytes stimulated with TG02 showed an increased CPM as compared to unstimulated splenocytes, indicating that TG02 induced an immune response.

TABLE 34
Counts per minute (CPM) after stimulation with TG02.

| | | | Splenocytes + |
|---|---|---|---|
| | Splenocytes | Splenocytes + TG02 | ConA |
| Mouse #4 | 12825 | 7772 | 15,113 |
| | 2540 | 10,038 | 12,612 |
| | 2729 | 5313 | 17,906 |
| Mean Mouse #4 | 2635 | 7708 | 15,210 |
| Mouse #7 | 1541 | 3999 | 5305 |
| | 2116 | 1734 | 6252 |
| | 378 | 2721 | 6916 |
| Mean Mouse #7 | 1345 | 2818 | 6158 |
| Mouse #24 | 3061 | 11,673 | 7834 |
| | 2805 | 9547 | 8578 |
| | 2580 | 11,338 | 10,057 |
| Mean Mouse #24 | 2815 | 10,853 | 8823 |
| Mouse #29 | 1183 | 4616 | 9480 |
| | 8133 | 4840 | 13,292 |
| | 1573 | 1990 | 10,033 |
| Mean Mouse #29 | 1728 | 4728 | 10,935 |
| Mouse #30 | 12,136 | 9787 | 32,205 |
| | 3204 | 26,083 | 35,668 |
| | 4189 | 18,800 | 42,149 |
| Mouse #30 | 6510 | 18,223 | 36,687 |

### Example 3

In this Example, Buffy coats were collected from three normal donors (Buffy 5, Buffy 6, and Buffy 7) for testing of T cell responses to selected peptides reflecting exon 2, 3 and 4 mutations in RAS. The standard operating procedure (SOP) for monitoring of T cell responses in clinical studies with KRAS peptide vaccination (TG01) was used for testing of peptide cocktail TGX3 and individual peptides.

### Method

Test Peptides

TABLE 35
Peptides in peptide cocktail TGX3

| Peptides | Amino acid sequence | SEQ ID NO: |
|---|---|---|
| A146T | GIPFIETSTKTRQRVED | 1 |
| G13C | KLVVVGAGCVGKSALTI | 25 |
| G13D | KLVVVGAGDVGKSALTI | 26 |
| Q61R | LDILDTAGREEYSARD | 35 |

Peptide cocktail TGX3 was a mixture of equimolar amounts of A146T+G13C+G13D+Q61R.

Equipment/Reagents
Hettich Rotina 420 (radius 210) or equivalent
KOJAIR Silverline Blue Series laminar flow hood or equivalent
CO₂ incubator, Forma Scientific Model 3111 or equivalent
Water bath 37° C.
KOVA Glasstic slide (Cat no. 87144E, Hycor Biomedical Inc, Garden Grove, USA)
TopCount, Microplate scintillation counter (Packard Instrument Company, Meriden, USA)
Cell Harvester Filtermate 196 Harvester, (Packard Instrument Company, Meriden, USA)
Unifilter GF/C (Cat.no. 6-005174, Nerliens Meszansky, Oslo, Norway) or equivalent
Microscint-0 scintillation liquid (Cat. No. 6013611, Nerliens Meszansky, Oslo, Norway) or equivalent
Topseal-A (Cat. No. 6005185, Nerliens Meszansky, Oslo, Norway) or equivalent
³H-Thymidine (Cat no. ART178-D, Nerliens Meszansky, Oslo, Norway) or equivalent
CellGro DC medium (Cat. no. 0020801-0500, CellGenix GmbH, Freiburg, Germany) or equivalent
RPMI-1640 (Cat no: E15-840) PAA Labs, Linz, Austria) or equivalent
Dimethylsulfoxide (DMSO) (Cat no: D5879-500ML, Sigma-Aldrich Norway AS, Oslo, Norway) or equivalent
Mucomyst (Cat.no. 019249, Meda AS, Asker, Norway) or equivalent
Recombinant human interleukin-2 (IL-2, Proleukin®), (Chiron Therapeutics, Emeryville, USA) or equivalent
4-(2-hydroxyethyl)-1-piperazineethanesulfonic acid (HEPES) buffer 1M (Cat. No. S11-001, Fisher Scientific AS, Oslo, Norway) or equivalent
IL-7 (Cat no. 207-IL-025, R & D Systems Europe Ltd, Abingdon, UK) or equivalent
Gentamicin 40 mg/ml (Cat. No. Sanofi Aventis Norge AS, Lysaker, Norway) or equivalent
Human Serum Albumin 20%, (Cat. No. SW2G0013, Baxter AS, Oslo, Norway) or equivalent
24-well tissue culture plates (Cat. No. 734-1605, VWR International AS, Oslo, Norway) or equivalent
Microplate 96-well, round bottomed (Cat. No. 734-1797, VWR International AS, Oslo, Norway) or equivalent

Complete Cel/Gro DC Medium Used for Culture

This is CellGro DC medium to which the following is added to 500 ml medium for the final concentrations:

Gentamicin 50 ug/ml (stock is 40 mg/ml, add 630 microL of stock to 500 mL medium) Mucomyst 1.6 mg/ml (stock is 200 mg/ml, add 4 mL of stock to 500 mL medium) HEPES buffer 0.01M (stock is 1 M, add 5 ml of stock to 500 mL of medium)

Experimental Procedure

a) Isolation of PBMCs from Buffy Coats

The procedure was performed at room temperature. All handling of cells in the open was done in a vertical laminar flow hood.
1. 3 Buffy coats were ordered from Blodbanken, Ulleval, OUS, Norway
2. PBMCs were isolated according to methods C001-003 (platelet-low).
3. The number of viable cells was counted using KOVA Galsstic slides and the cell concentration adjusted to 4×10⁶ cells/ml in complete CellGro DC medium (see recipe). Total cell numbers: Buffy 5-585×10⁶, Buffy 6-360×10⁶, and Buffy 7-405×10⁶ cells.

b) First Stimulation—Bulk Cultures for Increasing Number of TGX3 and Peptide Reactive T-Cells
1. Transfered 1 ml of the different PBMCs (4×10⁶ cells/ml in DC medium) to each well in a 24-well plate.

TABLE 36
First Stimulation - Number of cells in bulk cultures

| | Total number of | | Peptide |
|---|---|---|---|
| | cells | TGX3-mix | A146T |
| Buffy 5 | 120 × 10⁶ | 10 | 10 |
| Buffy 6 | 120 × 10⁶ | 10 | 10 |
| Buffy 7 | 120 × 10⁶ | 10 | 10 |

2. Added TGX3-mix/single peptide to each well for a final concentration of 10 μM of each peptide.
3. Cultured the cells in a humidified incubator at 37° C./5% CO₂ for 3 days
4. Day 3: Added a final concentration of 20 iU/ml of recombinant human interleukin-2 (rIL-2, b#77) and final concentration of 5 ng/ml recombinant human IL-7 (b#25) to the cell cultures and continued incubation at 37° C./5% CO₂. This depended on how fast the cells were growing and needed to be decided by a trained person with T-cell culture experience (according to checklist for training, Section for Cell Therapy).
5. Days 4-12: Cells were checked regularly under the microscope and split when required (500 μl was withdrawn from each well and replaced with 500 μl fresh CellGro DC medium, supplemented with 20 iU/ml IL-2 and 5 ng/ml IL-7) and wells with slow growing cells were mixed together.

c) Second Stimulation (Day 14) of Bulk Cultures
1. Day 14: Harvested, washed and counted T-cells in the bulk cultures from 6.2;

TABLE 37
Total T cell numbers in cultures

| | | Peptide |
|---|---|---|
| | TGX3-mix | A146T |
| Buffy 5 | 8.1 × 10⁶ | 4 × 10⁶ |
| Buffy 6 | 5.4 × 10⁶ | 4 × 10⁶ |
| Buffy 7 | 13.5 × 10⁶ | 6.8 × 10⁶ |

Bulk culture cells (ca 2×10⁶ T cells/well) were re-stimulated once more with autologous irradiated (30 Gy) APCs (ca 2×10⁶ APCs/well) added their respective peptides (as described in step b) above).
2. Cultured the cells in a humidified incubator at 37° C./5% CO₂ for 3 days.
3. Day 3 after second stimulation (i.e. Day 17 of the procedure): A final concentration of 20 iU/ml of recombinant human interleukin-2 (rIL-2, b#77) and final concentration of 5 ng/ml recombinant human IL-7 (b#25) were added to the cell cultures and continued incubation at 37° C./5% CO₂ (as described in step b) above).
4. Day 18-26: Cells were checked regularly under the microscope and split when required (500 μl was withdrawn from each well and replaced with 500 μl fresh CellGro DC medium, supplemented with 20 iU/ml IL-2 and 5 ng/ml IL-7) and wells with slow growing cells were mixed together (see step b) above).

d) Third Stimulation (Day 19) and Testing for Specificities in 3-Day T-Cell Proliferation Assay—Bulk Cultures
1. Day 19: Harvested, washed and counted T-cells in the bulk cultures from 6.3;

TABLE 38
Total T cell numbers

| | | Peptide |
|---|---|---|
| | TGX3-mix | A146T |
| Buffy 5 | 12.6 × 10⁶ | 10.8 × 10⁶ |
| Buffy 6 | 9 × 10⁶ | 12.6 × 10⁶ |
| Buffy 7 | 23.4 × 10⁶ | 14.4 × 10⁶ |

2. Transferred 5×10⁴ T-cells from bulk cultures per well in flat-bottomed 96-well plates.
3. Thawed 1-2 vials of autologous PBMCs sample in CellGro DC medium. Irradiated PBMCs (30 Gy), count and 5×10⁴ cells were added to each well and stimulated with their respective peptides.
4. Added 5×10⁴ T cells to their respective wells (see Table 39)

TABLE 39
Proliferation Assay Layout Buffy 5, 6 and 7

| | | | | | | | | | | | | |
|---|---|---|---|---|---|---|---|---|---|---|---|---|
| 1 | 2 | 3 | 4 | 5 | 6 | 7 | 8 | 9 | 10 | 11 | 12 | Culture |

| | | | |
|---|---|---|---|
| A | APC* | T cells | TGX3- |
| B | APC* + T cells | APC* + T cells + | mix |
| | | TGX3-mix | |
| C | APC* + T cells + | APC* + T cells + | |
| | peptide G13C | peptide G13D | |
| D | APC* + T cells + | APC* + T cells + | |
| | peptide Q61R | peptide A146T | |
| E | T cells | APC* + T cells | A146T |
| | | APC* + T cells + | |
| F | | peptide A146T | |

APC*—irradiated PBMC (Day 23)

5. Third Stimulation: Remaining cells were re-stimulated (ca 2×10⁶ T cells/well) once more with irradiated autologous APCs (2 mill/well) added their respective peptide (as described 6.2).
6. Day 21: Added 20 μL ³H-Thymidine (3.7×10⁴ Bq).
7. Incubated at 37° C./5% CO₂ for 17 hours
8. Day 22: Harvested the cells to Unifilters using the Filtermate 196 Harvester and dried the filters at 45° C. until completely dry (normally this is achieved after 1.5 but the number of hours left at 45° C. after this is not critical, hence plates can be counted 60 hours later).
9. Covered the bottom of the Unifilters with adhesive covers (delivered with the Unifilters) and add 25 μl micro scintillation liquid to each well. Covered the plate with TopSeal and placed the filters in a TopCount Packard microplate scintillation beta counter: Entered assay wizard program. Selected protocol/program ³H Thymidine in triplicates. Entered report definition and ASCII file output.

Results

The results of the T-cell proliferation assays with the peptide mixture are shown in FIGS. 11 and 12. In particular, FIG. 11 shows that a peptide having a mutation at position 146 of the RAS protein is immunogenic and stimulates the induction of T-cells. FIG. 12 shows that the peptide mixture TGX3 is effective in inducing a T-cell response, not only to the peptide mixture, but also to the individual peptides in the peptide mixture. Thus, FIG. 12 shows, for the first time, that a peptide mixture comprising peptides each having a position 13, a position 61 or a position 146 point mutation can induce T-cell responses.

1. A method of treatment and/or prophylaxis of cancer, the method comprising the step of administering a peptide to a patient in need thereof, wherein said peptide is suitable for eliciting an immune response, wherein said peptide comprises a region which corresponds to a fragment of the RAS protein, wherein
said region comprises at least 8 amino acids which include a mutated position,
said region has at least 6 amino acid residues, other than at said mutated position, which are identical to the corresponding region of the RAS protein,
said region has an amino acid substitution at said mutated position, and
said mutated position is selected from the group consisting of position 117 and 146 of the RAS protein.

2. A method according to claim 1, wherein the amino acid substitution is selected from the group consisting of a K117N, a A146T or a A146V substitution.

3. (canceled)

4. A method of treatment and/or prophylaxis of cancer, the method comprising administering a peptide mixture to a patient in need thereof, wherein said peptide mixture is suitable for eliciting an immune response and comprises a first and a second peptide, each corresponding to a fragment of the RAS protein wherein:
the first peptide comprises a region of at least 8 amino acids which includes a first mutated position,
the second peptide comprises a region of at least 8 amino acids which includes a second mutated position,
each of said regions of the first and second peptides independently has at least 6 amino acid residues, other than at said first and second mutated positions, which are identical to the corresponding region of the RAS protein,
each of the first and second peptides has an amino acid substitution at said first and second mutated positions,
wherein the first mutated position is selected from the group consisting of position 117 and 146 of the RAS protein and the second mutated position is selected from the group consisting of position 12, 13, 61, 117 and 146 of the RAS protein,
and wherein the amino acid substitution of the first mutated position is different from the amino acid substitution of the second mutated position.

5. A method according to claim 4, wherein the amino acid substitution of the first peptide is selected from the group consisting of a K117N, a A146T and a A146V substitution.

6. A method according to claim 4, wherein the amino acid substitution of the second peptide is selected from the group consisting of a K117N, a A146T, a A146V, a G13A, G13C, G13D, G13R, G13S, a G13V, G12A, G12C, G12D, G12R, G12S, a G12V, Q61E, Q61H, Q61K, Q61L, a Q61P and a Q61R substitution.

7. A method according to claim 4, wherein the peptide mixture comprises at least one further peptide corresponding to a fragment of the RAS protein, wherein:
said at least one further peptide comprises a region of at least 8 amino acids which includes a mutated position,
said region of said at least one further peptide has at least 6 amino acid residues, other than at said mutated position, which are identical to the corresponding region of the RAS protein,
said at least one further peptide has an amino acid substitution at said mutated position,
wherein said mutated position of the at least one further peptide is selected from the group consisting of position 12, 13, 61, 117 and 146 of the RAS protein, and
the amino acid substitution of said at least one further peptide is different from the amino acid substitution of each of the first and second RAS peptides.

8. A method according to claim 4, wherein the first mutated position is position 146 of the RAS protein and the second mutated position is selected from the group consisting of position 12, 13 and 61 of the RAS protein.

9. A method according to claim 7, wherein the first mutated position, the second mutated position and the mutated position of the at least one further peptide are selected from the group consisting of:
(i) the first mutated position is position 146 of the RAS protein, the second mutated position is position 12 of the RAS protein and the mutated position of the at least one further peptide is position 13 of the RAS protein,
(ii) the first mutated position is position 146 of the RAS protein, the second mutated position is position 13 of the RAS protein, and the mutated position of the at least one further peptide is position 61 of the RAS protein,
(iii) the first mutated position is position 146 of the RAS protein, the second mutated position is position 12 of the RAS protein, a first further peptide having a mutated position which is position 13 of the RAS protein, and a second further peptide having a mutated position which is position 61 of the RAS protein, and
(iv) the first mutated position is position 146 of the RAS protein, the second mutated position is position 13 of the RAS protein, the first further peptide has a mutated position which is position 13 of the RAS protein, and the peptide mixture further comprises a second further peptide having a mutated position which is position 61 of the RAS protein.

10-12. (canceled)

13. A method according to claim 4, wherein the peptide mixture comprises:
a peptide having a A146T substitution,
a peptide having a G13R substitution,
a peptide having a G13V substitution,
a peptide having a Q61R substitution,
a peptide having a Q61K substitution,
a peptide having a Q61H substitution, and
a peptide having a Q61L substitution.

14. A method of treatment and/or prophylaxis of cancer, wherein the method comprises the step of administering, to a patient in need thereof, one selected from the group consisting of:
a) at least one T-cell specific for the peptide defined in claim 1, when presented on an MHC molecule;
b) a T-cell preparation comprising a T-cell as defined in a) above;
c) a T-cell receptor or antigen-binding fragment thereof specific for a peptide as defined in claim 1, when presented on an MHC molecule;
d) a nucleic acid comprising a nucleotide sequence which encodes a peptide as defined in claim 1 or a T-cell receptor or antigen-binding fragment thereof specific for a peptide as defined in claim 1;
e) a vector comprising a nucleic acid as defined in d) above;
f) a host cell comprising a vector as defined in e) above; and
g) a pharmaceutical composition comprising a pharmaceutically acceptable carrier, diluent and/or excipient and one selected from the group consisting of a peptide as defined in claim 1, a T-cell as defined in a) above, a T-cell preparation as defined in b) above, a T-cell receptor or antigen-binding fragment as defined in c) above, a nucleic acid as defined in d) above, a vector as defined in e) above and a host cell as defined in f) above.

15-25. (canceled)

26. A method of treatment of cancer according to claim 1, wherein the cancer is selected from the group consisting of adrenal gland, autonomic ganglia, biliary tract, bone, breast, central nervous system, cervical, colorectal, endometrial, haematopoietic, lymphoid, kidney, large intestine, liver, lung, oesophagus, ovarian, pancreatic, prostate, salivary gland, skin, small intestine, stomach, testicular, thymus, thyroid, upper aerodigestive tract and urinary tract cancer and malignant melanoma.

27. A method of treatment or prophylaxis of cancer according to claim 26, wherein the cancer is colorectal cancer.

28. A method of treatment and/or prophylaxis of cancer, wherein the method comprises the step of administering, to a patient in need thereof, one selected from the group consisting of:
a) a T-cell mixture comprising T-cells specific for each of the peptides in one of the peptide mixtures as defined in claim 4, when presented on an MHC molecule; and
b) a pharmaceutical composition comprising a pharmaceutically acceptable carrier, diluent and/or excipient and a T-cell mixture as defined in a) above.
